(19) United States
(12) Patent Application Publication
(10) Pub. No.: US 2009/0012033 A1
(43) Pub. Date: Jan. 8, 2009

(54) Delivery of Biologically Active Materials Using Core-Shell Tecto(Dendritic Polymers)

(76) Inventors: Cordell R. DeMattei, Mt. Pleasant, MI (US); Baohua Huang, Mt. Pleasant, MI (US); Lori A. Reyna, Midland, MI (US); Sonke Svenson, Midland, MI (US); Douglas R. Swanson, Mt. Pleasant, MI (US); Donald A. Tomalia, Midland, MI (US); Michael A. Zhuravel, Mt. Pleasant, MI (US); Veera Reddy Pulgam, Mt. Pleasant, MI (US)

Correspondence Address: TECHNOLOGY LAW, PLLC, 3595 N. SUNSET WAY, SANFORD, MI, 48657, US

(21) Appl. No.: 12/224,644

(22) PCT Filed: Mar. 3, 2007
(86) PCT No.: PCT/US2007/005681

§ 371 Date: Sep. 2, 2008

(51) Int. Cl. A61K 48/00; C08G 73/02; C08G 63/08; C12Q 1/68; A61P 43/00; C12N 15/63; C08G 63/91; A61K 31/7052

(52) U.S. Cl. 514/44; 528/367; 528/354; 525/54.2; 435/455; 435/6

(57) ABSTRACT

The present invention concerns core-shell tecto (dendritic polymers) that are associated with biologically active materials (such as nucleic acids for use for RNAi and in transfection). Also included are formulations for their use. The constructs are useful for the delivery of drugs to an animal or plant and may be in vivo, in vitro or ex vivo.

### FEDERALLY SPONSORED RESEARCH STATEMENT

This invention was made with Government support under DAAL-01-1996-02-044 and W911NF-04-2-0030 awarded by The Army Research Laboratory Contract by the Department of Defense. The Government has certain rights in this invention.

### BACKGROUND OF THE INVENTION

1. Field of the Invention

RNA interference (RNAi) or post-transcriptional gene silencing is a biological response to double-stranded RNA. Recently, small interfering RNA (siRNA) has been explored as an effective agent to silence gene expression (RNA interference). [See for example, Fire, A. et al. Nature 391, 806-811 (1998)]. RNA is processed into 21-22 nucleotide dsRNAs (siRNA) that are used by the cell to recognize and destroy complementary RNAs, inhibiting formation of the corresponding gene product. This technology is used for basic research purposes to analyze gene function through sequence-specific gene silencing as well as for pharma/therapeutic purposes, where siRNA is used for drug target discovery and validation and also silencing disease-causing genes.

In order to facilitate the transfer of siRNA into a cell, various transfer agents are being pursued. The transfer of DNA using dendrimers has been reported earlier. See Haensler, J., et al., Bioconjugate Chem. 4, 372-379 (1993); Kukowska-Latallo, J., et al., PNAS 93, 4897-4902 (1996); Bielinska, A., et al., Nucleic Acids Research 24, 2176-2182 (1996); and Hudde, T., et al., Gene Ther. 6, 939-943 (1999).

This invention relates to the synthesis of a bio-complex comprising a dendritic polymer and nucleic acid, stabilization of the nucleic acid, and the uptake of the bio-complex by cells. This process could be performed both in in vitro transfection and in vivo delivery of nucleic acids to target cells for the inhibition of gene expression.

2. Description of Related Art

Dendrimers are highly branched, often spherical molecules in which branches terminating at charged amino groups, such as with PAMAM dendrimers, radiate from a central core molecule. Due to controlled chemical synthesis, dendrimers have a very precise size and defined shape.

Polyamidoamine (PAMAM) dendrimers have been used as non-viral vectors for both in vitro, in vivo, and ex vivo delivery of DNA and oligonucleotides. [See for example U.S. Pat. No. 5,527,524 and Polymeric Gene Delivery:Principles and Applications, Chapt. 9, ed. Mansoor M. Amiji, CRC Press (2005).] These radially symmetrical branched polymers are water soluble, biocompatible, and elicit little to no immune response. Amine-terminated dendrimers have a high density of positively charged amine groups on the surface, facilitating their interaction with negatively charged nucleic acids. Stable dendrimer-DNA complexes result from the electrostatic interactions between the positively charged amine groups on the dendrimer surface and the negatively charged phosphate groups on the DNA backbone. Complexed with the dendrimer, the DNA is protected from nuclease activity [see Chen, W., et al., Langmuir 1, 15-19 (2000)], facilitating maximal gene expression upon entry into the cell.

For use of such dendrimers in transfection various methods have been used to improve their transfection efficiency. In one such method Tang, M. X. et al., [Bioconjugate Chem. 7, 703 (1996)] disclosed a method of activation of these dendrimers that involves removal of some of the tertiary amines, resulting in a molecule with a higher degree of flexibility. These activated dendrimers yield a transfection efficiency 2-3 orders of magnitude higher than non-activated dendrimers. It is believed that these activated dendrimers assemble DNA into compact structures through the interaction of negatively charged phosphate groups of nucleic acids with the positively charged amino groups of the dendrimers. The resulting activated-dendrimer-DNA complexes possess a net positive charge that enables binding to the negatively charged surface molecules of the cell membrane. The transfection complexes are taken up by nonspecific endocytosis. The reagent buffers the pH of the endosome, leading to pH inhibition of endosomal nucleases, which ensures stability of the activated-dendrimer-DNA complexes. The defined size and shape of dendrimers ensures consistent transfection-complex formation and reproducibility of transfection results. QIAGEN offers two activated-dendrimer reagents for efficient and reproducible transfection of cells with DNA—PolyFect™ and SuperFect™ m Transfection Reagents. These reagents offer significant advantages over classical transfection technologies, such as higher transfection efficiencies, the ability to perform transfection in the presence of serum, and low cytotoxicity.

Haensler, J., et al., Bioconjugate Chem. 4, 372-379 (1993) were the first to demonstrate PAMAM dendrimer-mediated transfection of cell cultures. Using luciferase or galactosidase reporter plasmids with PAMAM dendrimers (G2-G10), as vectors, they investigated the transfection efficiency of both adherent and suspension cultured cells, including primary cell cultures. Adherent cell lines were represented by CV-1 (monkey fibroblast), HeLa (human carcinoma), and HepG2 (human hepatoma) cells; suspension cell cultures were represented by K-562 (human erythroleukemia), EL4 (mouse lymphoma), and Jurkat (human T-cells) cells. Rat hepatocytes were used as a primary cell culture model. Cells from all groups could be transfected (using G=6 PAMAMs), however certain cells showed better expression than others. For example, CV-1 and K-562 cells exhibited from 30-80% and 10-30% transfection, respectively, while EL-4 and Jurkat cells showed less than 1% transfection. This result was not surprising since most transfection systems display cell selectivity; however the molecular mechanisms for this variability remain unclear. Finally, transfection efficiency was determined to be directly related to the size of the dendrimer and dendrimer/DNA charge ratio. Luciferase expression increased up to 3 orders of magnitude by increasing the dendrimer diameter from 4 nm to 5.4 nm (G=4 to G=5, respectively), and maximal expression was obtained using G=6 dendrimers (6.8 nm diameter) in CV-1 cells. A dendrimer/DNA ratio of 6:1 (6 terminal amines to 1 phosphate) was shown to have optimal transfection efficiency, whereas higher ratios resulted in less efficiency.

An extensive investigation into the transfection properties of several series of intact monodispersed dendrimers was performed on a variety of cells by Kukowska-Latallo, J., et al., PNAS 93, 4897-4902 (1996). This group used both NH₃ and EDA core PAMAM dendrimers and studied the transfection efficiencies of G=0-10 in 18 different cell lines, ranging from rat fibroblasts to human lymphoma cells. G=3-10 dendrimers were shown to form stable complexes with DNA. However, only G=5 to G=10 exhibited significant cell transfection properties with a plateau occurring after G=8. Spherical shape and increase in surface charge were thought to be responsible for these effects. Overall, the PAMAM dendrimers were capable of transfecting many different cell types, including Jurkat and primary human fibroblasts, which are typically difficult to transfect, with no specific generation optimal for every type.

In addition to plasmid transfections, PAMAM dendrimers were also demonstrated to be effective vectors for oligonucleotide delivery [See Bielinska, A., et al. Nucleic Acids Research 24, 2176-2182 (1996); Yoo, H. et al., Nucleic Acids Research 28, 4225-4231 (2000); Delong, R. et al., J. Pharm. Sci. 1997, 86, 762-764 (1997); Axel, D. I., et al., J. Vasc. Res. 2000, 37, 221-234 (2000)].

Bielinska and co-workers were the first group to report dendrimers as anti-sense oligonucleotide transfection agents [Bielinska, A., et al., Nucleic Acids Research 24, 2176-2182 (1996)]. Luciferase expression in stably transfected Rat-2 fibroblasts and D5 mouse melanoma cells was maximally inhibited by ˜50% using PAMAM G=7-antisense oligonucleotides complexed at a 10:1 charge ratio. Using radiolabeled oligonucleotides, the amount of radiolabeled DNA in U937 human histiocytic lymphoma, Rat-2, D5, and Jurkat cells was 300 times greater when complexed with G=5, 7, and 9 dendrimers. After 24 hrs of transfection, PAMAM (G=7)/oligonucleotide-transfected cells still showed ˜75% anti-sense inhibition of luciferase expression compared to 100% expression in uncomplexed transfected cells. Not only did dendrimers facilitate oligonucleotide delivery, but they also appeared to extend oligonucleotide intracellular effectiveness by increasing stability.

Recent reports of successful in vivo dendrimer based vector experiments support the potential future use of dendrimers in therapeutic applications. One study reported dendrimer-mediated gene therapy for prostate cancer [Nakanishi, H., et al., Gene Ther. 10, 434-442 (2003)]. Prostate cancer-derived tumors were established in severe combined immunodeficiency mice. Intratumoral injections of dendrimer complexed with Fas ligand plasmid, a death ligand important in initiating apoptosis, resulted in the apoptosis of the tumor cells and significant growth suppression of the tumors. Another group reported the use of angiostatin and tissue inhibitor of metalloproteinase (TIMP-2) genes in an attempt to inhibit tumor growth and angiogenesis. [See Vincent, L., et al., Int. J. Cancer 105, 419-429 (2003).] Intratumoral injection of dendrimers complexed with angiostatin or TIMP-2 plasmids significantly inhibited tumor growth by 71% and 84%, respectively, and transfection combining the two plasmids resulted in growth inhibition by 96%. These data support the viable use of dendrimer-mediated therapeutic gene delivery in animal models.

U.S. Pat. No. 5,527,524 discloses the use of dendrimers to carry genetic material. Aggregates of dendrimers and mixture of sizes of dendrimers were tested for use as carriers. No testing of the present core-shell tecto(dendritic polymers) was disclosed.

Currently the products available on the market are cytotoxic to many cell types, have low transfection efficiencies, and lack targeting capabilities. Thus there is a need for a product the overcomes these issues.

### BRIEF SUMMARY OF THE INVENTION

The core-shell tecto(dendritic polymer) structures of the present invention possess several unique components that manifest surprising properties (compared to traditional dendritic structures) for RNAi. Low toxicity, protection from nucleases, and efficiency of transfer mediated by dendrimers makes them an excellent nucleic acid delivery vehicle. This invention refers to transfer of nucleic acids into cells, especially for the purpose of RNAi.

The present invention concerns a core-shell tecto(dendritic polymer) structure of the formula:

[C−(TF)ₙ]*[S-(TF)ₘ]ₓ Formula I

wherein:
[C] is the core dendritic polymer having (TF) groups present;
(TF) means a terminal functionality, which, if n is greater than 1, then (TF) may be the same or a different moiety;
n means the number of surface groups from 1 to the theoretical number possible for [C];
[S] is the shell dendritic polymer having (TF) groups present;
(TF) means a terminal functionality, which, if m is greater than 1, then (TF) may be the same or a different moiety;
m means the number of surface groups from 1 to the theoretical number possible for [S];
x means the number of [S] entities that surround [C] which are from 1 to the theoretical number possible for the (TF) present on [C];
* means a covalent bond; and
provided that both [C] and [S] may not be simultaneously PAMAM.

When the formulation and method of this invention are discussed both [C] and [S] for Formula I above may be PAMAM.

### BRIEF DESCRIPTION OF THE DRAWINGS

FIG. 1 illustrates the reaction of a nucleophilic core dendrimer with an excess of electrophilic shell dendrimer reagent to produce a partial shell filled tecto(dendrimer), which can be further reacted with a capping agent to form a hydroxyl surface partial shell filled tecto(dendrimer). [FIG. 12(b), Materials Today, 43, March 2005.]

FIG. 2 illustrates two routes to partial shell filled tecto(dendrimers). Route I involves amidation of a limited amount of nucleophilic core dendrimer with an excess of electrophilic shell dendrimer reagent to produce reactive (PS:CST)-[D_{c}-N—X]-amide-{Dₛ-E-Y}ₙ. These products may be pacified by reacting with 2-aminoethanol (EA) or tris (hydroxymethyl)aminomethane (TRIS) to produce shell pacified-(PS:CST)-[D_{c}-N—X]-amide-{Dₛ-E-Z}ₙ. Route II involves amidation of a limited amount of electrophilic core dendrimer with an excess of nucleophilic shell dendrimer reagent to produce reactive-(PS:CST)-[D_{c}-E-Y]-amide-{Dₛ-N—X}ₙ. These products may be converted to pacified forms by reacting with 2-aminoethanol (EA) or an excess of ethylenediamine (EDA) to produce core pacified-(PS:CST)-[D_{c}-E-Z]-amide-{Dₛ-N—X}ₙ. [FIG. 8, PNAS, 99(8), 5085, Apr. 16, 2002.] These shell reagents may also be dendrons.

FIG. 3 shows the results of testing two of the core-shell tecto(dendrimers) of Formula I for PPIB knockdown. In the HEK 293 cell line both core-shell tecto(dendrimers) showed significant knockdown compared to Lipofectamine™. In the MDCK cell line only the G=5(G=3 TREN) core-shell tecto(dendrimer) showed knockdown of the target gene, PPIB.

FIG. 4 shows the results of testing various core-shell tecto(dendrimers) of Formula I as siRNA delivery vehicles at varying concentrations in HEK 293 cells and MDCK cells. The results show that as the amount of surface positive charge increases, toxicity increases and that the larger the size of the core-shell tecto(dendrimer) used, the lower the transfection efficiency (the G=6 cores). The smaller core-shell tecto(dendrimers) used showed good transfection efficiency [G=4EA(G=3 NH₂)].

FIG. 5 shows the results from transfecting HEK 293 cells using PAMAM core-shell tecto(dendrimers) of Formula I. All tested core-shell tecto(dendrimers) showed results as good as or better than Lipofectamine (61%). On the x-axis the numbers following the tested material indicate the concentration used in μg/mL.

FIG. 6 shows the results from transfecting MDCK cells using PAMAM core-shell tecto(dendrimers) of Formula I. All tested core-shell tecto(dendrimers) showed results considerably better than Lipofectamine (27%). On the x-axis the numbers following the tested material indicate the concentration used in μg/mL.

FIG. 7 shows the results from transfecting HEK 293 and MDCK cells using PEHAM core-shell tecto(dendrimers) of Formula I. On the x-axis the numbers following the tested material indicate the concentration used in μg/mL.

### DETAILED DESCRIPTION OF THE INVENTION

### Glossary

The following terms as used in this application are to be defined as stated below and for these terms, the singular includes the plural.
ACTB (β-Actin, Genospectra, Inc.)
AEP means 1-(2-aminoethyl)piperazine
APS ammonium peroxydisulfate
Aptamer means a specific synthetic DNA or RNA oligonucleotide that can bind to a particular target molecule, such as a protein or metabolite
Backbone means the phosphate and the sugar groups of the nucleic acid
BL means blocking solution
BSA means bovine serum albumin
CE means capture extender solution
Celite means diatomaceous earth (Fisher Scientific)
Cyclophilin B is a target gene
DAB means diaminobutane
DCM means dichloromethane
DEIDA means diethyliminodiacetate
DI water means deionized water
DMAc means dimethylacetamide
DMF means dimethylforamide
DMI means dimethylitaconate
DMSO means dimethylsulfoxide; from Acros organics and further distilled prior to use
DTT means dithiothreitol
EA means ethanolamine or 2-aminoethanol
EDA means ethylenediamine; Aldrich
EDTA means ethylenediaminetetraacetic acid
EHTBO means 1-ethyl-4-(hydroxymethyl)-2,6,7-trioxabicyclo-[2.2.2]-octane equiv. means equivalent(s)
Et means ethyl
EtOH mean ethanol
FBS means fetal bovine serum
G means dendrimer generation, which is indicated by the number of concentric branch cell shells surrounding the core (usually counted sequentially from the core)
g means gram(s)
HCl means hydrochloric acid
HEK Cells means human embryonic kidney cells; HEK 293 is a specific cell line
Hexanes means mixtures of isomeric hexane (Fisher Scientific)
IMDA means iminodiacetic acid diethyl ester
IR means infrared spectrometry
L means liter(s)
LE means lead extender solution
Lipofectamine means Lipofectamine™ 2000 (Invitrogen Corporation)
LNA means locked nucleic acid
mA means milliamphere(s)
MALDI-TOF means matrix-assisted laser desorption ionization time of flight mass spectroscopy
MDCK Cells means Madin-Darby canine kidney cells
Me means methyl
MEM means Modified Eagle's Medium (Fischer Scientific)
MeOH means methanol
mg means milligram(s)
MIBK means methylisobutylketone
Mins. means minutes
mL means milliliter(s)
mock means a control transfection protocol where no siRNA is included in the transfection
MTT means 3-(4,5-Dimethyl-2-thiazolyl)-2,5-diphenyl-2H-tetrazolium bromide
NMR means nuclear magnetic resonance
ns means non-specific siRNA (Dharmacon, Inc.)
N—SIS means nanoscale sterically induced stoichiometry
OAc means acetate
PAGE means poly(acrylamide) gel electrophoresis
PAMAM means poly(amidoamine), including linear and branched polymers or dendrimers with primary amine terminal groups
PBS means phosphate buffered saline
PEHAM means poly(etherhydroxylamine) dendrimer
PEI means poly(ethyleneimine)
PETGE means pentaerythritol tetraglycidyl ether
Percent or % means by weight unless stated otherwise
PIPZ means piperazine or diethylenediamine
PNA means peptide nucleic acid
POPAM means a PPI core surrounded by PAMAM dendrons
PPI means poly(propyleneimine)
PPIB means peptidyl prolyl isomerase B (Genospectra, Inc.)
PPT means pentaerythritol propargyl triglycidyl ether
PVDF means polyvinylidene fluoride
R_{f} means relative flow in TLC
RT means ambient temperature or room temperature, about 20-25° C.
SDS means sodium dodecylsulfate
SIS means sterically induced stoichiometry
siTox means siCONTROL Tox siRNA (Dharmacon, Inc.)
TBE means tris(hydroxymethyl)amidomethane, boric acid and EDTA disodium buffer
TBS means TRIS-buffered saline
TE means 10 mM TRIS, 1 mM EDTA
TEA means triethyl amine
THF means tetrahydrofuran
TLC means thin layer chromatography
TMPTGE means trimethylolpropane triglycidyl ether; Aldrich; first distilled and purified by column chromatography (1.75′×10′) over silica gel (200-400 mesh) with 1:2:2 ratio of hexanes, ethyl acetate and chloroform as elutes. Purification of 5 g of TMPTGE gave 3.2 g (64% yield) of pure (>98%) material. Reaction was kept for 60 hours as precaution or done overnight.
TREN means tris(2-aminoethyl)amine
TRIS means tris(hydroxymethyl)aminomethane
Tween means polyoxyethylene (20) sorbitan mono-oleate
UF means ultrafiltration
UV-vis means ultraviolet and visible spectroscopy

This invention describes the synthesis of dendritic polymer/nucleic acid complexes, stabilization of the nucleic acid by the dendritic polymer, and uptake of the dendritic polymer/nucleic acid complexes by cells. Stable dendritic polymer/nucleic acid complexes result from the electrostatic interactions between the positively charged groups on the polymer surface and the negatively charged phosphate groups on the nucleic acid. Complexed with the dendritic polymer, the nucleic acid is protected from degradation, facilitating efficient delivery of the nucleic acid into the cell. This method for delivering nucleic acids is intended for RNAi applications including, but not limited to, basic research purposes to analyze gene function, drug target discovery and validation, and silencing genes for therapeutic purposes.

Also this invention describes the use of the core-shell tecto(dendritic polymers) of Formula I as delivery agents for biologically active materials other than nucleic acids. Examples of such biologically active materials include, but are not limited to, pro-drugs, pharmaceuticals, small organic molecules, and biomolecules. Additionally these core-shell tecto(dendritic polymers) of Formula I may be formulated with usual excipients, and other inert ingredients for administration.

### Chemical Structures

The core-shell tecto(dendritic polymer) structures of the present invention possess several unique components that manifest surprising properties (compared to traditional dendritic structures) for use in delivery of nucleic acids (in vivo, in vitro, or ex vivo). A structure for these dendritic polymers is shown by Formula I below:

[C-(TF)ₙ]*[S-(TF)ₘ]ₓ Formula I
wherein:
[C] is the core dendritic polymer having (TF) groups present;
(TF) means a terminal functionality, which, if n is greater than 1, then (TF) may be the same or a different moiety;
n means the number of surface groups from 1 to the theoretical number possible for [C];
[S] is the shell dendritic polymer having (TF) groups present;
(TF) means a terminal functionality, which, if m is greater than 1, then (TF) may be the same or a different moiety;
m means the number of surface groups from 1 to the theoretical number possible for [S];
x means the number of [S] entities that surround [C] which are from 1 to the theoretical number possible for the (TF) present on [C];
* means a covalent bond; and
provided that both [C] and [S] may not be simultaneously PAMAM.

When the formulation and method of this invention are intended, however, both [C] and [S] for Formula I may be PAMAM. [C] and [S] may be any dendritic polymer, including without limitation, PAMAM dendrimers, PEHAM dendrimers, PEI dendrimers, POPAM dendrimers, PPI dendrimers, polyether dendrimers, dendrigrafts, dendrons, random hyperbranched dendrimers, polylysine dendritic polymers, arborols, cascade polymers, or other dendritic architectures. There are numerous examples of such dendritic polymers in the literature, such as those described in Dendrimers and other Dendritic Polymers, eds. J. M. J. Frechet, D. A. Tomalia, pub. John Wiley and Sons, (2001) and other such sources.

[C] and [S] may be the same or different dendritic polymer structures both for class of components and for dendritic composition. These [C] and [S] dendritic polymers can be any physical shape, such as for example spheres, rods, tubes, or any other shape possible. The interior structure of either [C] or [S] or both may have an internal cleavable bond (such as a disulfide). Additionally, [S] can be a dendron. This dendron can have any dendritic polymer constituents desired as for [S].

Additionally, [C] may also comprise moieties that are size comparable and able to be functionalized and react with [S-(TF)] groups. Examples of such pseudo-dendritic polymers are: functionalized latex particles and hyperbranched polymers; quantum dots (e.g., CdSe, CdS, Au, Cu, etc.), functionalized fullerenes, carbon nanotubes, diamondoids [J. E. Dahl et al., Science 229, 96-99 (Jan. 3, 2003)]; colloidal silica; and macrocyclics (e.g., cellulose, sugars, carbohydrates, polyvinyl alcohols; crown ethers, etc.). The preferred size range for these pseudo-dendritic polymers is from about 10 nm (about G=10 PAMAM) to about 1,000 nm.

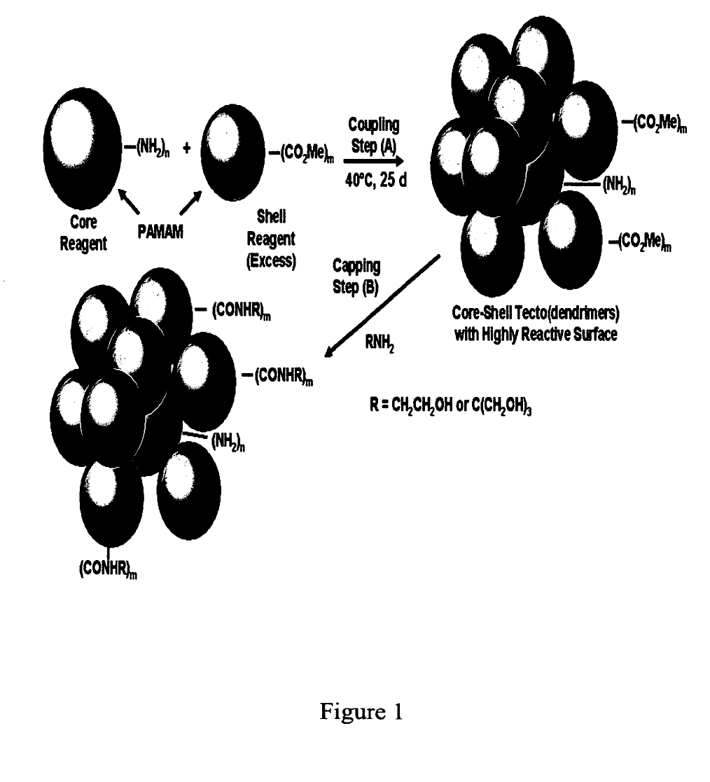

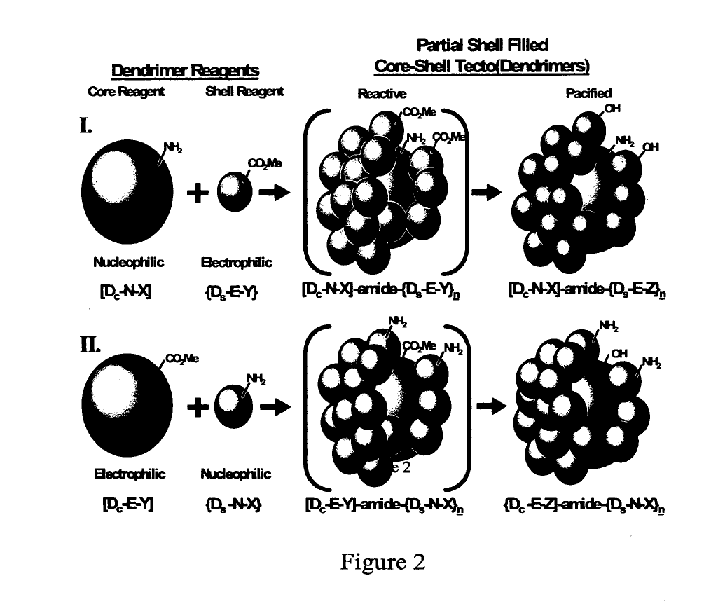

The (TF) groups on each of [C] and [S] must have at least 1 or more groups on each of [C] and [S] that can react between [C] and [S] to form a covalent bond, shown by * in Formula I. Additionally when [S] is a dendron, then the focal functionality (FF) of the dendron may react with the (TF) of [C]. For example, [C] can have some of its (TF) groups as primary amines from a PAMAM that react with the [S] (TF) groups that are carboxylic acids or esters (e.g., ethyl esters) in the presence of DCC forms an amide as the covalent bond of Formula I. See FIGS. 1 and 2.

When [C] is a PEHAM dendrimer with at least one (TF) as an epoxy group and [S] is a dendron with a focal functionality (FF) of sulfhydryl, the desired product of Formula I forms with a thioether as the covalent bond. When at least one the (TF) groups of [C] is an oxazoline and at least one of the (TF) groups of [S] is carboxylic acid, then an esteramide forms the covalent bond.

Any combination of (TF) groups capable of forming a covalent bond between [C] and [S] may be used. Thus one (TF) surface may have electrophilic moieties and the other (TF) surface would have nucleophilic moieties. Also the (FF) of a dendron may react with the (TF) of a [C] in a similar manner. The reaction conditions would be well known to those skilled in the art of organic synthesis. Some preferred examples of such (TF) groups are: amine-carboxylic acid; amine carboxylic ester; azide-acetylene groups; SH—SH for disulfide bonds; and amine-epoxide.

The number of [S] that can theoretically fit in the space available around [C] is indicated by the number x. While not wishing to be bound by theory, it is believed that the constraints are determined by N—SIS. When the sterics of the [S] exceeds the [C] physical space, then there will be unreacted (TF), i.e., nascent functionality. This nascent space can then be occupied by the nucleic acid or other biologically active materials for various advantages such as to protect it from degradation, and/or increase in the amount of carried material. If (TF) is a nascent amine(s), they are removed from contact with the cells so the toxicity of the core-shell tecto(dendritic polymer) is lowered.

Core-shell tecto(dendrimers) of Formula I where [C] and [S] are both PAMAM dendrimers are described in U.S. Pat. No. 6,635,720. These reaction mechanisms can be applied to other dendritic polymers having similar surface (TF) entities.

Formula I above also includes the use of a low generation dendrimer (e.g., sphere, rod, or any other shape dendrimer) then covering its surface with low-generation dendrons as [S] entities (i.e., G=1 or G=2) by chemical linkage. This approach not only allows preparation of a product with molecular weight similar to that of a higher generation dendrimer (i.e., G=4) in one step but also creates a product with enhanced purity compared to a ‘traditional’ G=4 dendrimer since the level of defects in low generation dendrimers is lower than the level of defects in higher generation dendrimers.

The dendronized dendrimers can be composed of any of the possible dendritic polymers or pseudo-dendritic polymers. Some examples are PAMAM core and dendron shell, PAMAM core with PEHAM dendron shell, PEHAM core with PAMAM dendron shell and PEHAM core with PEHAM dendron shell. In addition, dendronized dendrimers with mixed PAMAM and PEHAM dendron shells can be prepared. In addition, dendrons can be analogues of PAMAM such as polyether dendrons. All shell dendrons can either have the same terminal functionality (TF) or different dendrons can have different (TF), resulting in the formation of heterogeneous dendronized dendrimers. Furthermore, the length of branches, branching density (i.e., using AB₂ AB₃ etc. branching reagents) for dendritic polymers and additionally internal functionality (IF) (e.g., OH, SH, NH₂, COOH etc.) can be different for PEHAM-based dendrons. These dendronized polymers behave like the core-shell tecto(dendrimers) and are a part of Formula I as core-shell tecto(dendritic polymers). The dendronized shell will impart container properties to the product and make it amenable for drug encapsulation.

### General Syntheses of [C] or [S] for Use in Formula I

Most of these dendritic polymers have been taught in the literature. See Dendrimers and other Dendritic Polymers, eds. J. M. J. Frechet, D. A. Tomalia, pub. John Wiley and Sons, (2001) where most of these structures are discussed. The synthesis of the PEHAM structures of Formula II has been taught in WO/2006/115547, published Nov. 2, 2006, in detail from pp 37-58; particularly described below is the synthesis taught at pp 23-24, 46 and 50-51.

When [C] and/or [S] is a PEHAM dendritic polymer it has the following general formula

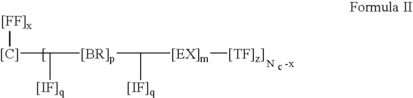

wherein:
(C) means a core;
(FF) means a focal point functionality component of the core;
x is independently 0 or an integer from 1 to N_{c}-1;
(BR) means a branch cell, which, if p is greater than 1, then (BR) may be the same or a different moiety;
p is the total number of branch cells (BR) in the dendrimer and is an integer from 1 to 2000 derived by the following equation

$$\begin{matrix}p = Total \# of [ BR ] \\ = ( \frac{{N}_{b}^{1}}{{N}_{b}} + \frac{{N}_{b}^{2}}{{N}_{b}} + \frac{{N}_{b}^{3}}{{N}_{b}} + … \frac{{N}_{b}^{G}}{{N}_{b}} ) [ {N}_{c} ] \\ = ( {\sum }_{i = 0}^{i = G - 1} {N}_{b}^{i} ) [ {N}_{c} ]\end{matrix}$$

where:
   G is number of concentric branch cell shells (generation) surrounding the core;
   i is final generation G;
   N_{b} is branch cell multiplicity; and
   N_{c} is core multiplicity and is an integer from 1 to 1000;
(IF) means interior functionality, which, if q is greater than 1, then (IF) may be the same or a different moiety;
q is independently 0 or an integer from 1 to 4000;
(EX) means an extender, which, if m is greater than 1, then (EX) may be the same or a different moiety;
m is independently 0 or an integer from 1 to 2000;
(TF) means a terminal functionality, which, if z is greater than 1, then (TF) may be the same or a different moiety;
z means the number of surface groups from 1 to the theoretical number possible for (C) and (BR) for a given generation G and is derived by the following equation

z=N_{c}N_{b}^{G};
where: G, N_{b} and N_{c} are defined as above; and
with the proviso that at least one of (EX) or (IF) is present.

Certain PEHAM structures of Formula II are prepared by an acrylate-amine reaction system which comprises:
A. Reacting an acrylate functional core with an amine functional extender, such as shown below:

(C)+(EX)→(C)(EX)(TF)
where (C)=an acrylate functional core such as TMPTA; (EX)=an amine functional extender such as PIPZ; and (TF)=amine; and
B. Reacting an amine functional extended core reagent of (C) (EX) (TF1) with an acrylate functional branch cell reagent (BR) as shown below:

(C)(EX)(TF1)+(BR)→(C)(EX)(BR)(TF2)
where (C)=TMPTA; (EX)=PIPZ; (TF1)=Amine; (BR)=TMPTA; and (TF2)=Acrylate; and
wherein for both Steps A and B
   the addition of an extender (EX) group to a core, the mole ratio of (EX)/(C) is defined as the moles of extender molecules (EX) to the moles of reactive functional groups on the simple core, scaffolding core, super core, or current generation structure (i.e. N_{c}) where an excess of (EX) is used when full coverage is desired;
   the addition of a branch cell (BR) to a simple core, scaffolding core, super core, or current generation structure (BR)/(C) is defined as the moles of branch cell molecules (BR) to the moles of reactive functional groups on the simple core, scaffolding core, super core, or current generation structure (i.e. N_{c}) where an excess of (BR) is used when full coverage is desired; and
   the level of addition of branch cells (BR) or extenders (EX) to a core, scaffolding core, super core or current generational product can be controlled by the mole ratio added or by N—SIS.

Another process to prepare the PEHAM dendritic polymers of Formula II as defined above is by a ring-opening reaction system which comprises:
A. Reacting an epoxy functional core with an amine functional extender, such as shown below:

(C)+(EX)→(C)(IF1)(EX)(TF1)
where:
   (C)=an epoxy functional core such as PETGE;
   (IF1)=Internal hydroxyl (OH);
   (EX)=piperazine (PIPZ);
   (TF1)=Amine; and
B. Reacting an amine functional extended core reagent (C) (IF 1) (EX) (TF 1) with an epoxy functional branch cell reagent such as shown below:

(C)(IF1)(EX)(TF1)+(BR)→(C)(IF1)(EX)(IF2)(BR)(TF2)
where:
   (C)=PETGE;
   (IF1)=Internal functionality moiety as defined in Formula II such as OH; (EX)=an extender moiety as defined in Formula II such as PIPZ;
   (TF1)=Amine;
   (BR)=an epoxy functional branch cell reagent such as PETGE;
   (IF2)=Internal functionality moiety as defined in Formula II such as OH; and
   (TF2)=Amine; and
wherein for both Steps A and B
   the addition of an extender (EX) group to a core, the mole ratio of (EX)/(C) is defined as the moles of extender molecules (EX) to the moles of reactive functional groups on the simple core, scaffolding core, super core, or current generation structure (i.e. N_{c}) where an excess of (EX) is used when full coverage is desired;
   the addition of a branch cell (BR) to a simple core, scaffolding core, super core, or current generation structure (BR)/(C) is defined as the moles of branch cell molecules (BR) to the moles of reactive functional groups on the simple core, scaffolding core, super core, or current generation structure (i.e. N_{c}) where an excess of (BR) is used when full coverage is desired; and
   the level of addition of branch cells (BR) or extenders (EX) to a core, scaffolding core, super core or current generational product can be controlled by the mole ratio added or by N—SIS.

An orthogonal chemical approach has been described in WO/2006/115547, published Nov. 2, 2006, particularly at pp 55-58, which concerns the 1,3-dipolar cyclo-addition of azides containing (C) and (BR) to alkynes containing (C) and (BR). The alkyne containing (C) may have from 1 to N_{c} alkyne moieties present and alkyne containing (BR) may have from 1 to N_{b}-1 alkyne moieties. The other reactive groups present in (C) or (BR) can be any of the (BR) groups listed herein before. Azide containing (C) and (BR) are produced by nucleophilic ring-opening of epoxy rings with azide ions. Subsequent reaction of these reactive groups can provide triazole linkages to new (BR) or (TF) moieties using “click” chemistry as described by Michael Malkoch et al., in J. Am. Chem. Soc. 127, 14942-14949 (2005).

The desired utility for these core-shell tecto(dendritic polymers) of Formula I is to deliver nucleic acids in vivo, ex vivo or in vitro as a carrier to increase transfection, reduce toxicity and provide targeting. Thus (TF) may include targeting moieties, such as proteins, antibodies, synthetic molecules that are specific for the site for delivery. Also (TF) may include other moieties for use in detection of the conjugate (such as fluorescent entities, dyes, contrast agents, radionuclides, etc.), and/or for the treatment of a disease or condition and have conjugated to the surface, either by a chelant or directly, various pharmaceutical moieties, drugs, prodrugs, or other active entities. Because many of the core-shell (dendritic polymers) of Formula I have interior space available, they may also encapsulate the same or different entities as discussed above.

Thus the core-shell(dendritic polymers) of Formula I may have several different (TF) groups present on its surface. One method to prepare such (TF) groups is by reacting one desired (TF) with one of [S] or [C] and reacting another desired (TF) with the other [C] or [S] by selection of the surface reaction groups, and then forming the covalent bond. It is usually desired that the conjugate (Formula I and M) have an overall positive charge or partial positive charge to enable entry into the cell through the lipid bilayer. When the conjugate is used to transfect cells it may be administered to the cells by any of: standard incubation; electroporation; ballistic transfection; dermal; high pressure delivery (e.g., hydrodynamic tail vein injection); direct injection; or any other suitable method. These conjugates of this invention are believed useful for a variety of diseases, such as: cancer (e.g., proliferative, inflammatory, metabolic, autoimmune neurologic, ocular diseases); eclampsia; allergies; NMDA-R dysregulation disorders; Neurodegenerative diseases/disorders; Anti-viral agents (HepA,C; suppression of HepA translation/replication by targeting internal ribosomal entry site); Neurological disorders (by attenuating production of pro-inflammatory mediators); Respiratory viruses (RSV); Macular degeneration; Diabetic retinopathy; Alzheimer's disease; and AIDS. Additionally, this conjugate may be useful for:

nucleic acid delivery for treatment of other diseases caused by overexpression; for delivery of DNA or RNA to replace, by recombination into genome or direct expression from the construct, missing gene function; and/or for detection of genetic disease (i.e., a molecular beacon that only signals if it pairs to a disease causing gene).

The present conjugates (Formula I and M) have the advantages over known nucleic acid delivery systems because: the core-shell tecto(dendritic polymer) aids in protecting the nucleic acid from degradation; facilitates the entry into the cells, including use of enhancers; allows for targeting the conjugate by the (TF) groups; allows for the carrying of other moieties such as those that permit imaging to tell where the conjugate has gone in vivo; can be designed to enter cells and likely cross the blood-brain barrier; and have low toxicity compared to other known transfection agents.

The material is associated with the interior, surface or both the interior and surface of these dendritic polymers and the groups may be the same or different. As used herein “associated with” means that the carried material(s) (M) can be physically encapsulated or entrapped within the interior of the dendrimer, dispersed partially or fully throughout the dendrimer, or attached or linked to the dendrimer or any combination thereof, whereby the attachment or linkage is by means of covalent bonding, hydrogen bonding, adsorption, absorption, metallic bonding, van der Walls forces or ionic bonding, or any combination thereof. The association of the carried material(s) and the dendrimer(s) may optionally employ connectors and/or spacers or chelating agents to facilitate the preparation or use of these conjugates. Suitable connecting groups are groups which link a targeting director (i.e., T) to the dendrimer (i.e., D) without significantly impairing the effectiveness of the director or the effectiveness of any other carried material(s) (i.e., M) present in the combined dendrimer and material (“conjugate”). These connecting groups may be cleavable or non-cleavable and are typically used in order to avoid steric hindrance between the target director and the dendrimer; preferably the connecting groups are stable (i.e., non-cleavable) unless the site of delivery would have the ability to cleave the linker present (e.g., an acid-cleavable linker for release at the cell surface or in the endosomal compartment). Since the size, shape and functional group density of these dendrimers can be rigorously controlled, there are many ways in which the carried material can be associated with the dendrimer. For example, (a) there can be covalent, coulombic, hydrophobic, or chelation type association between the carried material(s) and entities, typically functional groups, located at or near the surface of the dendrimer; (b) there can be covalent, coulombic, hydrophobic, or chelation type association between the carried material(s) and moieties located within the interior of the dendrimer; (c) the dendrimer can be prepared to have an interior which is predominantly hollow (i.e., solvent filled void space) allowing for physical entrapment of the carried materials within the interior (void volume), wherein the release of the carried material can optionally be controlled by congesting the surface of the dendrimer with diffusion controlling moieties, (d) where the dendrimer has internal functionality groups (IF) present which can also associate with the carrier material, possesses a cleavable (IF) which may allow for controlled (i.e., pH dependent) exiting from the dendrimer interior or (e) various combinations of the aforementioned phenomena can be employed.

The material (M) that is encapsulated or associated with these dendrimers may be a very large group of possible moieties that meet the desired purpose. Such materials include, but are not limited to, pharmaceutical materials for in vivo or in vitro or ex vivo use as diagnostic or therapeutic treatment of animals or plants or microorganisms, viruses and any living system, which material can be associated with these dendrimers without appreciably disturbing the physical integrity of the dendrimer.

In a preferred embodiment, the carried materials, herein represented by “M”, are pharmaceutical materials. Such materials which are suitable for use in the present dendrimer conjugates include any materials for in vivo or in vitro use for diagnostic or therapeutic treatment of mammals which can be associated with the dendrimer without appreciably disturbing the physical integrity of the dendrimer, for example: drugs, such as antibiotics, analgesics, hypertensives, cardiotonics, steroids and the like, such as acetaminophen, acyclovir, alkeran, amikacin, ampicillin, aspirin, bisantrene, bleomycin, neocardiostatin, chlorambucil, chloramphenicol, cytarabine, daunomycin, doxorubicin, cisplatin, carboplatin, fluorouracil, taxol, gemcitabine, gentamycin, ibuprofen, kanamycin, meprobamate, methotrexate, novantrone, nystatin, oncovin, phenobarbital, polymyxin, probucol, procarbabizine, rifampin, streptomycin, spectinomycin, symmetrel, thioguanine, tobramycin, trimetoprim, and valbanl; toxins, such as diphtheria toxin, gelonin, exotoxin A, abrin, modeccin, ricin, or toxic fragments thereof; metal ions, such as the alkali and alkaline-earth metals; radionuclides, such as those generated from actinides or lanthanides or other similar transition elements or from other elements, such as ⁴⁷Sc, ⁶⁷Cu, ⁶⁷Ga, ⁸²Rb, ⁸⁹Sr, 88Y, ⁹⁰Y, ^{99m}Tc, ¹⁰⁵Rh, ¹⁰⁹Pd, ¹¹¹In, ^{115m}In, ¹²⁵I, ¹³¹I, ¹⁴⁰Ba, ¹⁴⁰La, ¹⁴⁹ Pm, ¹⁵³Sm, ¹⁵⁹Gd, ¹⁶⁶Ho, ¹⁷⁵Yb, ¹⁷⁷Lu, ¹⁸⁶Re, ¹⁸⁸Re, ¹⁹⁴Ir, and ¹⁹⁹Au, preferably ⁸⁸Y, ⁹⁰Y, ^{99m}Tc, ¹²⁵I, ¹³¹I, ¹⁵³Sm, ¹⁶⁶Ho, ¹⁷⁷Lu, ¹⁸⁶Re, ⁶⁷Ga, ¹¹¹In, ^{115m}In, and ¹⁴⁰La; signal generators, which includes anything that results in a detectable and measurable perturbation of the system due to its presence, such as fluorescing entities, phosphorescence entities and radiation; signal reflectors, such as paramagnetic entities, for example, Fe, Gd, or Mn; chelated metal, such as any of the metals given above, whether or not they are radioactive, when associated with a chelant; signal absorbers, such as near infrared, contrast agents (such as imaging agents and MRI agents) and electron beam opacifiers, for example, Fe, Gd or Mn; antibodies, including monoclonal or polyclonal antibodies and anti-idiotype antibodies; antibody fragments; aptamers; hormones; biological response modifiers such as interleukins, interferons, viruses and viral fragments; diagnostic opacifiers; and fluorescent moieties. Carried pharmaceutical materials include scavenging agents such as chelants, antigens, antibodies, aptamers, or any moieties capable of selectively scavenging therapeutic or diagnostic agents.

In another embodiment, the carried materials, herein represented by “M”, are agricultural materials. Such materials which are suitable for use in these conjugates include any materials for in vivo or in vitro treatment, diagnosis, or application to plants or non-mammals (including microorganisms) which can be associated with the dendrimer without appreciably disturbing the physical integrity of the dendrimer. For example, the carried materials can be toxins, such as diphtheria toxin, gelonin, exotoxin A, abrin, modeccin, ricin, or toxic fragments thereof; metal ions, such as the alkali and alkaline earth metals; radionuclides, such as those generated from actinides or lanthanides or other similar transition elements or from other elements, such as ⁴⁷Sc, ⁶⁷Cu, ⁶⁷Ga, ⁸²Rb, ⁸⁹Sr, ⁸⁸Y, ⁹⁰y, ^{99m}Tc, 105Rh, ¹⁰⁹Pd, ¹¹¹In, ^{115m}In, ¹²⁵I, ¹³¹I, ¹⁴⁰La, ¹⁴⁰La, ¹⁴⁹ Pm, ¹⁵³Sm, ¹⁵⁹Gd, 166Ho, ¹⁷⁵Yb, ¹⁷⁷Lu, ¹⁸⁶Re, ¹⁸⁸Re, ¹⁹⁴Ir, and ¹⁹⁹Au; signal generators, which includes anything that results in a detectable and measurable perturbation of the system due to its presence, such as fluorescing entities, phosphorescence entities and radiation; signal reflectors, such as paramagnetic entities, for example, Fe, Gd, or Mn; signal absorbers, such contrast agents and as electron beam opacifiers, for example, Fe, Gd, or Mn; hormones; biological response modifiers, such as interleukins, interferons, viruses and viral fragments; pesticides, including antimicrobials, algaecides, arithelmetics, acaricides, II insecticides, attractants, repellants, herbicides and/or fungicides, such as acephate, acifluorfen, alachlor, atrazine, benomyl, bentazon, captan, carbofuran, chloropicrin, chlorpyrifos, chlorsulfuron cyanazine, cyhexatin, cypermethrin, 2,4-dichlorophenoxyacetic acid, dalapon, dicamba, diclofop methyl, diflubenzuron, dinoseb, endothall, ferbam, fluazifop, glyphosate, haloxyfop, malathion, naptalam; pendamethalin, permethrin, picloram, propachlor, propanil, sethoxydin, temephos, terbufos, trifluralin, triforine, zineb, and the like. Carried agricultural materials include scavenging agents such as chelants, chelated metal (whether or not they are radioactive) or any moieties capable of selectively scavenging therapeutic or diagnostic agents.

In another embodiment, the carried material, herein represented by (M), are immuno-potentiating agents. Such materials which are suitable for use in these conjugates include any antigen, hapten, organic moiety or organic or inorganic compounds which will raise an immuno-response which can be associated with the dendrimers without appreciably disturbing the physical integrity of the dendrimers. For example, the carried materials can be synthetic peptides used for production of vaccines against malaria (U.S. Pat. No. 4,735,799), cholera (U.S. Pat. No. 4,751,064) and urinary tract infections (U.S. Pat. No. 4,740,585), bacterial polysaccharides for producing antibacterial vaccines (U.S. Pat. No. 4,695,624) and viral proteins or viral particles for production of antiviral vaccines for the prevention of diseases such as AIDS and hepatitis.

The use of these conjugates as carriers for immuno-potentiating agents avoids the disadvantages of ambiguity in capacity and structure associated with conventionally known classical polymer architecture or synthetic polymer conjugates used to give a macromolecular structure to the adjuvant carrier. Use of these dendrimers as carriers for immuno-potentiating agents, allows for control of the size, shape and surface composition of the conjugate. These options allow optimization of antigen presentation to an organism, thus resulting in antibodies having greater selectivity and higher affinity than the use of conventional adjuvants. It may also be desirable to connect multiple antigenic peptides or groups to the dendrimer, such as attachment of both T- and B-cell epitopes. Such a design would lead to improved vaccines.

Preferably the carried materials (M) are bioactive agents. As used herein, “bioactive” refers to an active entity such as a molecule, atom, ion and/or other entity which is capable of detecting, identifying, inhibiting, treating, catalyzing, controlling, killing, enhancing or modifying a targeted entity such as a protein, glycoprotein, lipoprotein, lipid, a targeted disease site or targeted cell, a targeted organ, a targeted organism [for example, a microorganism, plant or animal (including mammals such as humans)] or other targeted moiety. Also included as bioactive agents are genetic materials (of any kind, whether oligonucleotides, fragments, or synthetic sequences) that have broad applicability in the fields of gene therapy, siRNA, diagnostics, analysis, modification, activation, anti-sense, silencing, diagnosis of traits and sequences, and the like. These conjugates include effecting cell transfection and bioavailability of genetic material comprising a complex of a dendritic polymer and genetic material and making this complex available to the cells to be transfected.

These conjugates may be used in a variety of in vivo, ex vivo or in vitro diagnostic or therapeutic applications. Some examples are the treatment of diseases such as cancer, autoimmune disease, genetic defects, central nervous system disorders, infectious diseases and cardiac disorders, diagnostic uses such as radioimmunoassays, electron microscopy, PCR, enzyme linked immunoabsorbent assays, nuclear magnetic resonance spectroscopy, contrast imaging, immunoscintography, and delivering pesticides, such as herbicides, fungicides, repellants, attractants, antimicrobials or other toxins. Non-genetic materials are also included such as interleukins, interferons, tumor necrosis factor, granulocyte colony stimulating factor, and other protein or fragments of any of these, antiviral agents.

These conjugates may be formulated into a tablet using binders known to those skilled in the art. Such dosage forms are described in Remington's Pharmaceutical Sciences, 18^{th} ed. 1990, pub. Mack Publishing Company, Easton, Pa. Suitable tablets include compressed tablets, sugar-coated tablets, film-coated tablets, enteric-coated tablets, multiple compressed tablets, controlled-release tablets, and the like. Ampoules, ointments, gels, suspensions, emulsions, injections (e.g., intramuscular, intravenous, intraperitoneal, subcutaneous), transdermal formulation (e.g., patches or application to the skin surface, suppository compositions), intranasal formulations (e.g., drops, sprays, inhalers, aerosol spray, chest rubs), ocular application (e.g., sterile drops, sprays, ointments), or application in a gauze, wipe, spray or other means at site of surgical incision, near scar formation sites, or site of a tumor growth or removal, may also be used as a suitable formulation. Kits for bioassays as biomarkers, molecular probes are possible, including use with other reagents for the assay, and instructions for their use. Customary pharmaceutically-acceptable salts, adjuvants, binders, desiccants, diluents and excipients may be used in these formulations. For agricultural uses these conjugates may be formulated with the usual suitable vehicles and agriculturally-acceptable carrier or diluent, such as granular formulations, emulsifiable concentrates, solutions, and suspensions as well as combined with one or more than one active agent.

While not wishing to be bound by theory, it is believed that some of these advantages are caused by the core-shell tecto(dendritic polymer) of Formula I nano-clefts available to enclose or protect the M. See D. A. Tomalia, Materials Today 34-46 (March 2005) and D. A. Tomalia et al., PNAS 99(8), 5081-5087 (Apr. 16, 2002).

### General Syntheses of Conjugate

### Synthesis of Dendrimer-Nucleic Acid Complex—DNA Complexes

Incubation of plasmid DNA and dendrimers of Formula I for a minimum of 5 mins. at RT results in the formation of DNA/dendrimer complexes. The ratio of DNA to dendrimer is based on the electrostatic charge present on each component, which must be optimized for optimal gene delivery. [See Haensler, J., et al., Bioconjugate Chem. 4, 372-379 (1993); and Kukowska-Latallo, J., et al., PNAS 9, 4897-4902 (1996).]

### Synthesis of Dendrimer-Nucleic Acid Complex—Oligonucleotide Complexes

An aliquot of oligonucleotide at a given concentration is combined with various concentrations of dendrimer, mixed briefly, and allowed to incubate at RT for 5 mins. to allow complex formation. The ratio of oligo to dendrimer is based on the electrostatic charge present on each component, which must be optimized for optimal oligonucleotide delivery. [See Yoo, H. et al., Nucleic Acids Research 28, 4225-4231 (2000); and Bielinska, A., et al., Nucleic Acids Research 24, 2176-2182 (1996).]

### Synthesis of Dendrimer-Nucleic Acid Complex—RNA Complexes

The siRNA/dendrimer complexes will be formed using the same above methods, with buffers optimized for RNA. The ratio of RNA:dendrimer will have to be optimized as well. This method is further shown in the examples.

By the term “nucleic acids” (or “M”) this invention includes all forms of nucleic acid: single stranded (ss)DNA, RNA, PNA, LNA, and all double stranded (ds) combinations of these single stranded forms. Any source (synthetic or naturally isolated) and any length [from the smallest oligonucleotides (3 nucleotides) to whole chromosomes], including small hairpin RNA (shRNA), and aptamers. It also includes both unmodified and modified nucleic acids [on the backbone, bases, termini (3′ or 5′) and combinations of these modifications], where the sense and/or anti-sense strand nucleic acid are conjugated to the dendritic polymer. It would be possible and desired in some instances to have the anti-sense strand bound by other than covalent bonding and the sense strand bound by covalent bonding. The preferred number of nucleotides are from about 18-30, preferably from about 20-25.

The core-shell tecto(dendritic polymers) of Formula I are associated with one or more biologically active materials (“M”) to form a construct by ionic, electrostatic, van der Waals forces, covalent, or hydrogen bonding, including base-pairing. A transfection enhancing agent [e.g., fusogenic peptide (KALA), L-Arg conjugations] may be associated with the conjugate or separately present, when desired. The size of the conjugate of the core-shell tecto(dendritic polymers) of Formula I with M can be any size for the intended use, such as from 1-10,000 nm.

For the following examples the various equipment and methods were used to run the various described tests for the results reported in the examples described below.

### Equipment and Methods

### Size Exclusion Chromatography (SEC)

A methanolic solution of Sephadex™ (Pharmacia) purified dendrimer was evaporated and reconstituted with the mobile phase used in the SEC experiment (1 mg/mL concentration). All the samples were prepared fresh and used immediately for SEC.

Dendrimers were analyzed qualitatively by the SEC system (Waters 1515) operated in an isocratic mode with refractive index detector (Waters 2400 and Waters 717 Plus Auto Sampler). The analysis was performed at RT on two serially aligned TSK gel columns (Supelco), G3000PW and G2500PW, particle size 10 μm, 30 cm×7.5 mm. The mobile phase of acetate buffer (0.5M) was pumped at a flow rate of 1 mL/min. The elution volume of dendrimer was observed to be 11-16 mL, according to the generation of dendrimer.

### High Pressure/Performance Liquid Chromatography (HPLC)

High pressure liquid chromatography (HPLC) was carried out using a Perkin Elmer™ Series 200 apparatus equipped with refractive index and ultraviolet light detectors and a Waters Symmetry® C₁₈ (5 μm) column (4.6 mm diameter, 150 mm length). A typical separation protocol was comprised of 0.1% aqueous acetic acid and acetonitrile (75:25% v/v) as the eluant and UV light at λ=480 nm as the detector.

### Thin Layer Chromatography (TLC)

Thin Layer Chromatography was used to monitor the progress of chemical reactions. One drop of material, generally 0.05M to 0.4M solution in organic solvent, is added to a silica gel plate and placed into a solvent chamber and allowed to develop for generally 10-15 mins. After the solvent has been eluted, the TLC plate is generally dried and then stained (as described below). Because the silica gel is a polar polymer support, less polar molecules will travel farther up the plate. “R_{f}” value is used to identify how far material has traveled on a TLC plate. Changing solvent conditions will subsequently change the R_{f} value. This R_{f} is measured by the ratio of the length the product traveled to the length the solvent traveled.

Materials: TLC plates used were either (1) “Thin Layer Chromatography Plates—Whatman®” PK6F Silica Gel Glass backed, size 20×20 cm, layer thickness: 250 μm or (2) “Thin Layer Chromatography Plate Plastic sheets—EM Science” Alumina backed, Size 20×20 cm, layer thickness 200 μm.

Staining conditions were: (1) Ninhydrin: A solution is made with 1.5 g of ninhydrin, 5 mL of acetic acid, and 500 mL of 95% ethanol. The plate is submerged in the ninhydrin solution, dried and heated with a heat gun until a color change occurs (pink or purple spots indicate the presence of amine). (2) Iodine Chamber: 2-3 g of 12 is placed in a closed container. The TLC plate is placed in the chamber for 15 mins. and product spots will be stained brown. (3) KMnO₄ Stain: A solution is prepared with 1.5 g of KMnO₄, 10 g of K₂CO₃, 2.5 mL of 5% NaOH, and 150 mL of water. The TLC plate is submerged in KMnO₄ solution and product spots turn yellow. (4) UV examination: An ultraviolet (UV) lamp is used to illuminate spots of product. Short wave (254 nm) and long wave (365 nm) are both used for product identification.

### MALDI-TOF Mass Spectrometry

Mass spectra were obtained on a Bruker Autoflex™ LRF MALDI-TOF mass spectrometer with Pulsed Ion Extraction. Mass ranges below 20 kDa were acquired in the reflector mode using a 19 kV sample voltage and 20 kV reflector voltage. Polyethylene oxide was used for calibration. Higher mass ranges were acquired in the linear mode using a 20 kV sample voltage. The higher mass ranges were calibrated with bovine serum albumin.

Typically, samples were prepared by combining a 1 μL aliquot of a 5 mg/mL solution of the analyte with 10 μL of matrix solution. Unless otherwise noted, the matrix solution was 10 mg/mL of 2,5-dihydroxybenzoic acid in 3:7 acetonitrile:water. Aliquots (2 μL) of the sample/matrix solution were spotted on the target plate and allowed to air dry at RT.

### Dialysis Separation

In a typical dialysis experiment about 500 mg of product is dialyzed through a dialysis membrane with an appropriate pore size to retain the product and not the impurities. Dialyses are done in most examples in water (other appropriate dialyzates used were acetone and methanol) for about 21 hours with two changes of dialyzate. Water (or other dialyzate) is evaporated from the retentate on a rotary evaporator and the product dried under high vacuum or lyophilized to give a solid.

### Ultrafiltration Separation (UF)

A typical ultrafiltration separation protocol was as follows: A mixture of product and undesired compounds was dissolved in the appropriate volume of a solvent for this mixture (e.g., 125 mL of MeOH) and ultrafiltered on a tangential flow UF device containing 3K cut-off regenerated cellulose membranes at a pressure of 20 psi (137.9 kPa) at 25° C. The retentate volume as marked in the flask was maintained at 100-125 mL during the UF collection of 1500 mL permeate (˜5 hours). The first liter of permeate was stripped of volatiles on a rotary evaporator, followed by high vacuum evacuation to give the purified product. Depending on the specific separation problem, the cut-off size of the membrane (e.g., 3K, 2K or 1K) and the volume of permeate and retentate varied.

### Sephadex™ Separation

The product is dissolved in the minimum amount of a solvent (water, PBS, or MeOH) and purified through Sephadex™ LH-20 (Pharmacia) in the solvent. After eluting the void volume of the column, fractions are collected in about 2-20 mL aliquots, depending on the respective separation concerned. TLC, using an appropriate solvent as described before, is used to identify fractions containing similar product mixtures. Similar fractions are combined and solvent evaporated to give solid product.

### Nuclear Magnetic Resonance (NMR)—¹H and ¹³C

Sample preparation: To 50-100 mg of a dry sample was add 800-900 μL of a deuterated solvent to dissolve. Typical reference standards are used, i.e., trimethylsilane. Typical solvents are CDCl₃, CD₃OD, D₂O, DMSO-d₆, and acetone-d₆. The dissolved sample was transferred to an NMR tube to a height of ˜5.5 cm in the tube.

Equipment: (1) 300 MHz NMR data were obtained on a 300 MHz 2-channel Varian™ Mercury Plus NMR spectrometer system using an Automation Triple Resonance Broadband (ATB) probe, H/X (where X is tunable from ¹⁵N to ³¹P). Data acquisition was obtained on a Sun Blade™ 150 computer with a Solaris™ 9 operating system. The software used was VNMR v6.1C. (2) 500 MHz NMR data were obtained on a 500 MHz 3-channel Varian™ Inova 500 MHz NMR spectrometer system using a Switchable probe, H/X (X is tunable from ¹⁵N to ³¹P). Data acquisition was obtained on a Sun Blade™ 150 computer with a Solaris™ 9 operating system. The software used was VNMR v6.1C.

### Atomic Force Microscopy (AFM) or Scanning Probe Microscopy (SPM)

All images were obtained with a Pico-SPM™ LE AFM (Molecular Imaging, USA) in DI water with tapping mode, using Multi-purpose large scanner and MAC mode Tips [Type II MAClevers, thickness: 3 μm, length: 225 μm, width: 28 μm, resonance frequency: ca 45 KHz and force constant: ca 2.8 N/m (Molecular Imaging, USA)]. Typically, 3 lines/sec. scan speed was used for scanning different areas, with a set point of 0.90 of the cantilever oscillation amplitude in free status. To avoid hydrodynamic effect of thin air gaps, the resonance was carefully measured at a small tip—sample distance.

### Polyacrylamide Gel Electrophoresis (PAGE)

Dendrimers that were stored in solvent are dried under vacuum and then dissolved or diluted with water to a concentration about 100 mg in 4 mL of water. The water solution is frozen using dry ice and the sample dried using a lyophilizer (freeze dryer) (LABCONCO Corp. Model number is Free Zone 4.5 Liter, Freeze Dry System 77510) at about −47° C. and 60×10⁻³ mBar. Freeze dried dendrimer (1-2 mg) is diluted with water to a concentration of 1 mg/mL. Tracking dye is added to each dendrimer sample at 10% v/v concentration and includes (1) methylene blue dye (1% w/v) for basic compounds (2) bromophenol blue dye (0.1% w/v) for acid compounds (3) bromophenol blue dye (0.1% w/v) with 0.1% (w/v) SDS for neutral compounds.

Pre-cast 4-20% gradient gels were purchased from ISC BioExpress. Gel sizes were 100 mm (W)×80 mm (H)×1 mm (Thickness) with ten pre-numbered sample wells formed in the cassette. The volume of the sample well is 50 μL. Gels not obtained commercially were prepared as 10% homogeneous gels using 30% acrylamide (3.33 mL), 4×TBE buffer (2.5 mL), water (4.17 mL), 10% APS (100 μL), TEMED (3.5 μL). TBE buffer used for gel electrophoresis is prepared using tris(hydroxymethyl)aminomethane (43.2 g), boric acid (22.08 g), disodium EDTA (3.68 g) in 1 L of water to form a solution of pH 8.3. The buffer is diluted 1:4 prior to use.

Electrophoresis is done using a PowerPac™ 300 165-5050 power supply and BIO-RAD™ Mini Protean 3 Electrophoresis Cells. Prepared dendrimer/dye mixtures (5 μL each) are loaded into separate sample wells and the electrophoresis experiment run. Dendrimers with amine surfaces are fixed with a glutaraldehyde solutions for about one hour and then stained with Coomassie Blue R-250 (Aldrich) for about one hour. Gels are then destained for about one hour using a glacial acetic acid solution. Images are recorded using an hp Scanjet™ 5470C scanner.

### Infrared Spectrometry (IR or FTIR)

Infrared spectral data were obtained on a Nicolet Fourier™ Transform Infrared Spectrometer, Model G Series Omnic, System 20 DXB. Samples were run neat using potassium bromide salt plates (Aldrich).

### Ultraviolet/Visible Spectrometry (UV/Vis)

UV-VIS spectral data were obtained on a Perkin Elmer™ Lambda 2 UV/VIS Spectrophotometer using a light wavelength with high absorption by the respective sample, for example 480 or 320 nm.

siRNA Methods

### Transfection

Lyophilized dendrimers were brought up to 250 μL in MEM (10% FBS). In a separate Eppendorf tube, Cyclophilin B siRNA [Human PPIB; siGENOME duplex (Dharmacon, Inc.)] was brought up to 250 μL in MEM (10% FBS) for a final concentration of 150 nM. Both were allowed to incubate at RT for 15 mins. before mixing together and incubating for an additional 20 mins. Another 500 μL of media was added to each tube after incubation, bringing the total volume to 1 mL. This mixture was then added to 85% confluent HEK 293 or MDCK cells whose media had been completely aspirated. The cells were incubated with the dendrimer-siRNA complexes for 6 hours before replacing with fresh media. The cells were fed 48 hours later, and then harvested after 72 hours. The tissue culture plates were rinsed with PBS, then scraped in 150 μL of Western Lysis Buffer (15 mM of TRIS-HCL pH 7.4-8.0), 150 mM of NaCl, 1% of Triton X-100, and 1 mM of NaVO₄) and transferred to Eppendorf tubes. The samples were then vortexed and frozen at −20° C. until protein analysis.

Lipofectamine™ (Invitrogen Corporation) transfections were performed per the manufacturer's protocol, as directed for HEK 293 transfections. Basically, the same procedure as above was performed, however the media during complex formation was free from FBS and antibiotics. Complexes were formed with 2 μg/mL of Lipofectamine™.

### Protein Quantitation

Protein samples were thawed and vortexed, then centrifuged at 12K rpm. Samples were analyzed for protein content using the BioRad™ Protein Assay (BioRad) per manufacturer's protocol. Basically, 2 μL of protein sample were added to a 96 well microplate, followed by 200 μL of diluted BioRad™ reagent. The plate was read at 570 nm on a Multiskan MCC/340 microplate reader (ThermoLabsystems). BSA was used for the standard. Calculations were performed on the resulting data to determine protein quantitation of the samples.

### Western Blots

Twenty five micrograms of protein samples were run on 15%/5% SDS PAGE. The gels were run at 30 mA per gel. Following electrophoresis, the gels were assembled in a gel transfer apparatus and transferred to nitrocellulose membrane at 200 mA for 2 hours. The membranes were then removed, probed with Ponceau Red to monitor transfer efficacy, rinsed with TBS, and blocked in a 5% milk solution for 1 hour. After blocking, the membranes were incubated at RT with anti-Cyclophilin B antibody (1:3000 dilution) for 2 hours (Abcam, Inc.), followed by 2×5 min. rinses with TBS+0.05% Tween. Alkaline phosphatase-conjugated anti-rabbit secondary antibody (1:5000 dilution) was then incubated with the membranes for 1 hour, followed by 3×5 min. rinses with TBS+0.05% Tween. The membranes were then developed using 1-Step™ NBT/BCIP solution from Pierce. Images were captured digitally and analyzed for band density.

The invention will be further clarified by a consideration of the following examples, which are intended to be purely exemplary of the present invention. The lettered examples are synthesis of starting materials; the numbered examples are those examples of Formula I making core-shell tecto(dendritic polymers); and the Roman numbered examples are those examples of Formula I showing biological utility.

### Starting Materials

### A. Example A

### Ring-Opening Using an Diester amino Branch Cell Reagent Precursor

Ester Terminated PEHAM Dendrimer, G=1, from Trimethylolpropane Triglycidyl Ether (TMPTGE) and Diethyl iminodiacetate (DEIDA)
[(C)=TMPTGE; (FF)=Et; (IF1)=OH; (BR1)=DEIDA; (TF)=Ethyl ester; G=1.5]

DEIDA II (14.07 g, 74.47 mmol) (Aldrich) and 120 mL of dry MeOH were placed in an oven dried 250-mL single necked round bottom flask. The flask was equipped with a stir bar and septum. TMPTGE I (5.0 g, 16.55 mmol) (Aldrich) was dissolved in 40 mL of dry MeOH and then added to the above stirring solution through a pressure equalizing funnel dropwise over a period of one hour at RT. The funnel was replaced with refluxing condenser and the flask heated at 60° C. for 60 hours under a N₂ atmosphere. The solvent was removed on a rotary evaporator under reduced pressure, which gave a colorless transparent liquid. The entire reaction mixture was transferred into a 100-mL single necked round bottom flask. Excess of DEIDA II was removed by Kugelrohr distillation under reduced pressure at 150-160° C. Undistilled product III (12.59 g; 87.5% yield) was recovered as a pale yellow color, viscous liquid. Compound III is stored in EtOH at 0° C. Its spectra are as follows:

¹H NMR: (300 MHz, CD₃OD): δ 4.65 (sextet, J=4.20 Hz, 3H), 4.16 (m, 12H), 3.59 (s, 12H), 3.36 (s, 6H), 3.30 (s, 6H), 3.05 (dd, J=3.60 Hz, 3H), 2.95 (dd, J=3.90 Hz, 2H), 2.81 (dt, J=1.80 Hz & 9.90 Hz, 3H), 2.67 (dd, J=8.40 & 8.10 Hz, 2H), 1.37 (q, J=7.50 Hz, 2H), 1.26 (t, J=7.20 Hz, 6H, 2×CH₃), 1.25 (J=7.20 Hz, 12H, 6×CH₃), 0.85 (t, J=7.50 Hz, 3H, CH₃); and

¹³C NMR: (75 MHz, CD₃OD): δ 6.81, 13.36, 13.40, 22.66, 43.48, 49.85, 53.62, 55.76, 56.21, 58.00, 60.55, 60.68, 68.72, 71.17, 71.33, 71.50, 73.40, 78.43, 78.48, 168.67, 170.25, 172.31; and

IR (Neat): λₘₐₓ 2980, 2934, 2904, 2868, 1741, 1460, 1408, 1378, 1342, 1250, 1198, 1111, 1065, 1024, 983, 927, 860, 784 cm⁻¹; and

MALDI-TOF MS: C₃₉H₇₁N₃O₁₈ Calc. 869; found 893 (M⁺Na) and 847, 801, 779, 775 amu. (The mass spectrum shows a typical fragmentation pattern for elimination of OC₂H₅ group.)

The following Scheme A illustrates this reaction:

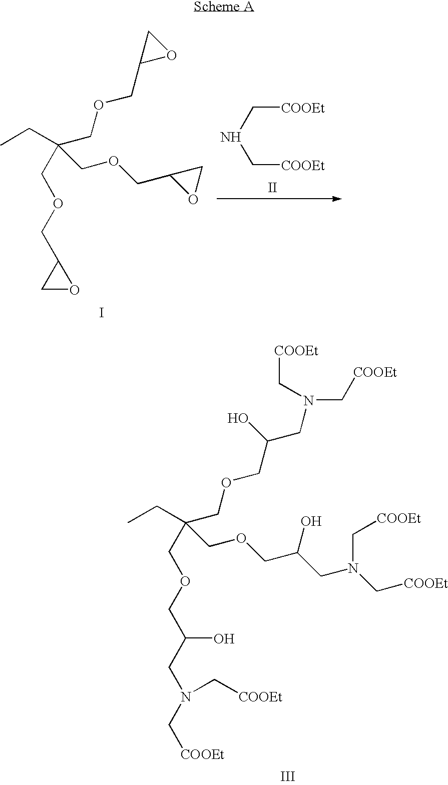

### EXAMPLE B

### Reaction of the product from trimethylolpropane triglycidylether reacting with diethyliminodiacetate (DEIDA) with tris(2-aminoethyl)amine (TREN) to produce PEHAM dendrimer G=2 with a three-arm core and primary amine surface

[(C)=TMPTGE; (FF)=Et; (IF 1)=OH; (BR1)=DEIDA; (BR2)=TREN; (TF)=Primary NH₂; G=2]

A 100-mL round bottom flask was charged with TREN 2 (17.05 g, 116.82 mmol, 60 NH₂ equiv. per ester) and 40 mL of MeOH (Fisher Scientific) and a magnetic stir bar. After the exothermic mixing reaction had stopped, (20 minutes), a solution of G=1 ester C4 (0.846 g, 0.97 mmol, 5.84 ester mmol; made from Example A) in 10 mL of MeOH was added dropwise over a period of 1 hour at RT. The mixture was then placed in an oil-bath and heated at 50° C. for 3 days. Progress of the reaction was monitored by IR spectroscopy, i.e., the disappearance of the ester vibration at 1740 cm⁻¹ and the appearance of the amide vibration at 1567 cm⁻¹. MALDI-TOF MS analysis indicated the mass for the desired G=2.0 product accompanied by looped compounds at 1348 [M+Na]⁺ and 1201 [M+Na]⁺ (one and two loops). The reaction mixture was diluted with 700 mL of MeOH and subjected to UF using a 1K size exclusion membrane. After collecting 1.8 liters of permeate, the retentate was withdrawn from the UF and the solvent removed by rotary evaporation, giving a pale yellow colored, viscous liquid, which was further dried under high vacuum to give the desired G=2 dendrimer 3 (1.41 g, 98.94% yield). Its spectra are as follows:

¹H NMR (300 MHz, CD₃OD): δ 0.86 (3H, bt), 1.38 (2H, bs), 2.32-2.60 (H, m), 2.67-2.76 (H, m), 3.29-3.34 (H, m), 3.82 (3H, bs); and

¹³C NMR (125 MHz, CD₃OD): δ 8.14, 24.06, 38.57, 38.63, 39.98, 40.16, 44.59, 54.00, 55.09, 55.28, 57.21, 58.02, 60.19, 63.05, 63.28, 69.38, 69.94, 72.52, 72.96, 75.00, 173.76, 173.86, 174.03; and

IR(Neat): νₘₐₓ 3298, 2934, 2842, 1659, 1572, 1536, 1470, 1388, 1357, 1311, 1116, 973, 819 cm⁻¹; and

MALDI-TOF MS: C₆₃H₁₄₃N₂₇O₁₂ Calc. 1470.9843; found 1494.2270 [M+Na]⁺, 1348.022 [M+Na]⁺ (one looped), 1201.0970 [M+Na]⁺ (two looped) amu.

The following Scheme B illustrates this reaction.

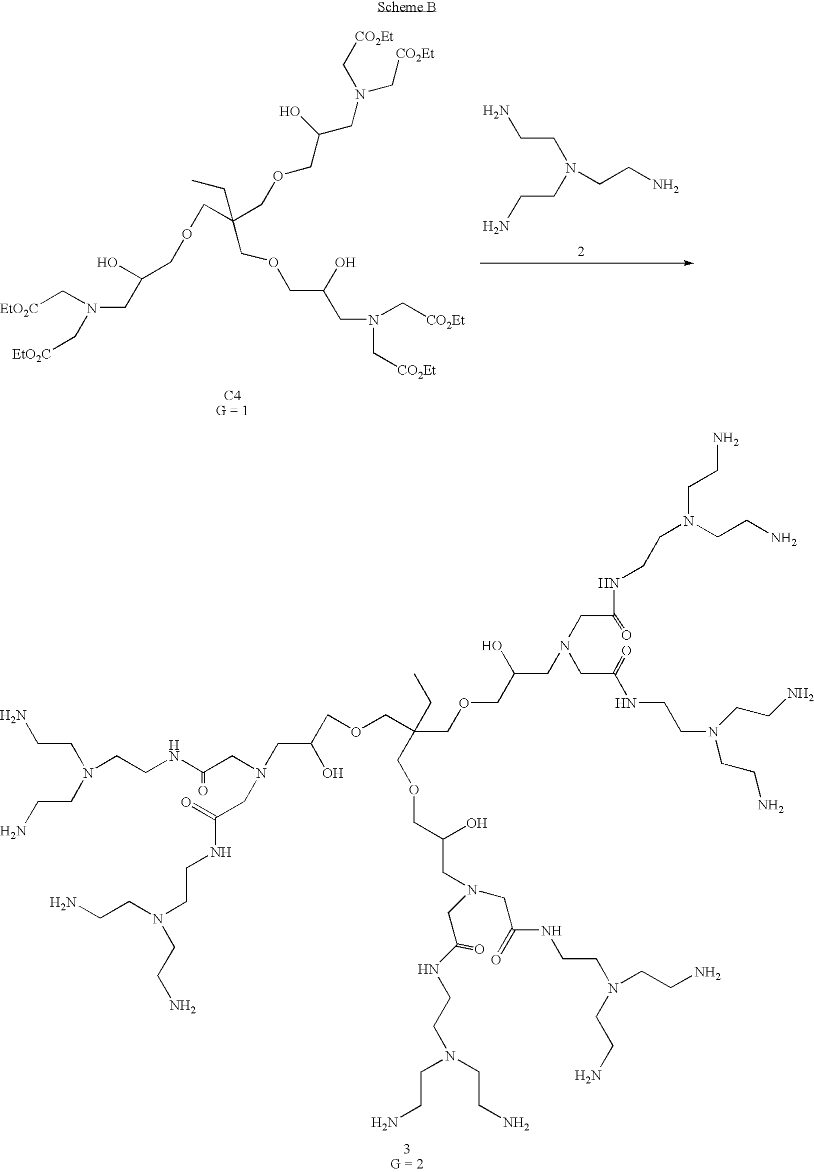

### EXAMPLE C

### Polyether Dendron G=0 with Tetra(Ethylene Glycol) Linker and Capped Hydroxyl (FF) and Hydroxyl (TF)

[(C)=Pentaerythritol; (FF)=O-Benzyl; (EX)=Tetra(ethylene glycol); (TF)=OH]
   A. Synthesis of Monoprotected Benzyloxy tetra(ethylene glycol)

A 250-mL round-bottom flask was plugged with a septum and purged with N₂ gas. Tetra(ethylene glycol) (49.11 g, 253.0 mmol) (Acros Organics) was weighed into the flask and dissolved in 70 mL of dry, degassed THF. Sodium hydride (2.02 g, 50.0 mmol, 0.2 equiv.) (Acros Organics) was weighed into 500-mL Schlenk flask, capped with septum and purged with N₂ gas. 100 mL of dry, degassed THF was added, and the slurry was cooled to −72° C. in a bath composed of dry ice and isopropanol. The tetra(ethylene glycol) solution was slowly added to the slurry via a cannula, and the reaction mixture was stirred until it started to freeze. The cooling bath was removed and the reaction mixture stirred for 1.5 hours at RT. Benzyl bromide (5.4 mL, 0.18 equiv.) (Aldrich) was added via a syringe to the clear solution, and the reaction mixture was stirred overnight. The solution was diluted to 400 mL with hexanes, and the solvent was removed by rotary evaporation. The residue was dissolved in water and extracted with DCM (2×100 mL). The combined organic extracts were dried over MgSO₄, the solution was filtered, the solvent removed by rotary evaporation, and the crude product purified by flash chromatography (1:1 EtOAc/acetone). Purification was followed by TLC (1:1 EtOAc/acetone), giving the product at R_{f}=0.60. The desired product was recovered as a clear oil (11.90 g; 92% yield). Its spectra are as follows:

¹H NMR (CDCl₃): δ 7.35-7.25 (m, 5H), 4.56 (2H), 3.71-3.57 (m, 16H), 3.08 (m, 1H); and

¹³ C NMR (CDCl₃): δ 138.2, 128.3, 127.8, 127.6, 73.2, 72.6, 70.6, 70.6-70.5 (m), 70.3, 69.4, 61.6; and

MALDI-TOF MS: C₁₅H₂₄O₅; calc. 284.2, found 307.5 [M+Na]⁺ amu.

The following Scheme C-A illustrates this reaction.

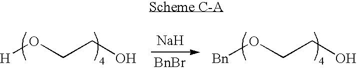

### Synthesis of benzyloxy tetra(ethylene glycol) tosylate

In a 500-mL round-bottom flask, benzyloxy tetra(ethyleneglycol) (8.87 g, 31.2 mmol) (made from Example C-A) and toluenesulfonylchloride (17.8 g, 93.6 mmol, 3 equiv.) (Aldrich) were dissolved in 100 mL of THF and cooled to 0° C. in an ice-bath. Then potassium hydroxide (14.9 g, 234.0 mmol, 7.5 equiv.) (Fisher Chemicals) dissolved in 100 mL of water was added dropwise over 20 min. After the addition was complete, the mixture was stirred for 1 hour at RT. The THF and water layers were separated, the aqueous layer extracted with 100 mL of EtOAc, and the combined organic fractions washed with brine (2×100 mL), then dried over MgSO₄, and filtered. The solvent was removed by rotary evaporation, and the product was dried under vacuum to give a clear oil (13.3 g; 97% yield). If desired, the product can be purified by flash chromatography (EtOAc, R_{f}=0.75). Its spectra are as follows:

¹H NMR (CDCl₃): δ 7.80-7.77 (m, 2H), 7.34-7.24 (m, 7H), 4.55 (2H), 4.15-4.13 (m, 2H), 3.68-3.55 (m, 16H), 2.43 (3H); and

¹³C NMR (CDCl₃): δ 144.6, 138.1, 132.9, 129.7, 128.2, 127.8, 127.6, 127.4, 73.1, 70.6, 70.5 (m), 70.4, 69.3, 69.1, 68.5, 21.5; and

MALDI-TOF MS: C₂₂H₃₀O₇S; calc. 438.2, found 461.2 [M+Na]⁺ amu.

The following Scheme C-B illustrates this reaction.

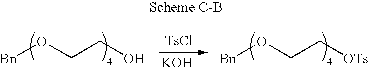

### C. Synthesis of EHTBO

A 250-mL round-bottom flask was charged with pentaerythritol (51.27 g, 377 mmol) (Acros Organics), triethylorthopropionate (67.04 g, 381.0 mmol, 1.01 equiv.) (Aldrich), and pyridinium p-toluenesulfonate (950.0 mg, 3.8 mmol, 0.01 equiv.) (Acros Organics). The flask was equipped with a Dean-Stark trap and a reflux condenser and heated with stirring to 130° C. Collection of ethanol as a byproduct of the reaction started at 120° C. and continued for 30 min. After the ethanol production had ended, the reaction was heated to 160° C. for 1 hour, then the Dean-Stark trap was replaced with a short-path distillation apparatus and the product was vacuum-distilled (bp=115° C., 5 mm Hg) to give the product, EHTBO, as colorless oil (62.1 g; 96% yield), which solidified on cooling to −20° C. Its spectra are as follows:

¹H NMR (CDCl₃): δ 3.94 (6H), 3.36 (d, 2H, J_{HH}=5), 2.60 (t, 1H, J_{HH}=5), 1.62 (q, 2H, J_{HH}=4=8), 0.88 (t, 3H, J_{HH}=8); and

¹³C NMR (CDCl₃): δ 110.0, 69.5, 61.2, 35.9, 30.0, 7.6.

The following Scheme C-C illustrates this reaction.

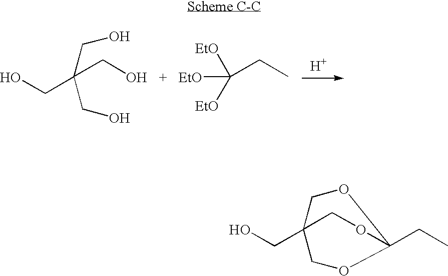

### D. Synthesis of benzyloxy tetra(ethylene glycol) G0-propionate

Benzyloxy tetra(ethylene glycol) tosylate (9.20 g, 21.0 mmol) was weighted into a 100-mL round-bottom flask, purged with N₂ gas and dissolved in 70 mL of dry, degassed THF. EHTBO (3.95 g, 1.1 equiv.) (made from Example C-C) was weighed in a 100-mL round-bottom flask, which was capped with a septum, and purged with N₂ gas. 50 mL of dry, degassed THF was added and the solution was cannula-transferred into a 250-mL Schlenk flask containing sodium hydride (1.15 g, 27.6 mmol, 1.25 equiv. to EHTBO) (Acros Organics). The resulting mixture was stirred for 1.5 hours at RT. To this mixture, the solution of benzyloxy tetra(ethylene glycol) tosylate was added via a cannula and the reaction allowed to stir for 16 hours. The reaction was quenched with MeOH by dilution to 300 mL volume, and the solvent was removed by rotary evaporation. The residue was dissolved in DCM and washed with 100 mL of water. The aqueous wash was extracted with 50 mL DCM and the combined organic fraction was dried over MgSO₄. The solvent was removed by rotary evaporation to give the crude benzyloxy tetra(ethylene glycol)-G=0-propionate as yellow oil (8.94 g; 100% yield). An analytical sample was set aside and purified by column chromatography (EtOAc, R_{f}=0.55). The bulk of the product was used immediately without further purification. Its spectra are as follows:

¹H NMR (CDCl₃): δ 7.35-7.25 (m, 5H), 4.56 (2H), 3.99 (6H), 3.69-3.57 (m, 14H), 3.52-3.49 (m, 2H), 3.22 (2H), 1.69 (q, 2H), 0.94 (t, 3H); and

¹³C NMR (CDCl₃): δ 138.1, 128.2, 128.0, 127.6, 127.4, 109.6, 73.0, 71.1, 70.6, 70.5, 70.3, 69.6, 69.3, 69.2, 63.1, 60.9, 35.6, 35.0, 29.7, 7.4; and

MALDI-TOF MS: C₂₃H₃₈O₉; calc. 458.3, found 481.2 [M+Na]⁺ amu.

The following Scheme C-D illustrates this reaction.

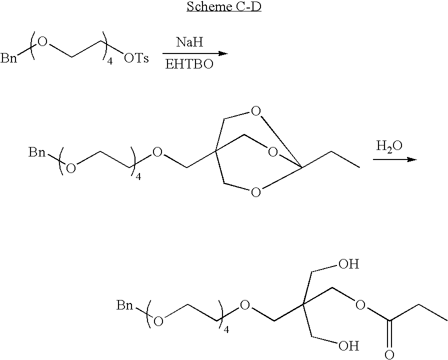

### E. Synthesis of G=0 dendron benzyloxy tetra(ethylene glycol)-G=0-OH

Crude benzyloxy tetra(ethylene glycol)-G=0-propionate was dissolved in 100 mL of MeOH. Then 4 mL of concentrated HCl were added and the solution stirred for 3 hours at 60° C. The solution was cooled to RT and the reaction quenched by addition of aqueous sodium hydrogen carbonate (NaHCO₃). The solvent was evaporated by rotary evaporation, the solid residue dissolved in DCM and washed with 100 mL of water. The aqueous wash was extracted with 50 mL of DCM and the combined organic fraction dried over MgSO₄. The solvent was evaporated by rotary evaporation and the product immediately used in the next step. An analytical sample was purified by column chromatography (1:1 DCM/acetone; R_{f}=0.30). Its spectra are as follows:

¹H NMR (CDCl₃): δ 7.35-7.25 (m, 5H), 4.55 (2H), 3.68-3.57 (m, 22H), 3.50 (2H), 3.18 (br, 3H); and

¹³C NMR (CDCl₃): δ137.9, 128.2, 128.1, 127.7, 127.5, 73.1, 71.6, 70.5, 70.4, 70.3, 70.1, 69.2, 63.5, 45.1; and

MALDI-TOF MS: C₂₀H₃₄O₈; calc. 402.2, found 425.2 [M+Na]⁺ amu.

The following Scheme C-E illustrates this reaction.

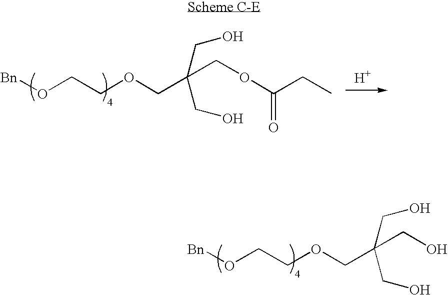

### EXAMPLE D

### Polyether Dendron G=1 with tetra(ethylene glycol) Linker and Hydroxyl (FF) and Methoxy (TF)

[(C)=Pentaerythritol; (FF)=OH; (EX)=Tetra(ethylene glycol); (BR)=Pentaerythritol; (TF)=OMe]
   A. Synthesis of benzyloxy tetra(ethylene glycol)-G=0-OTs

Into a 250-mL round-bottom flask capped with septum and purged with N₂ gas, 50 mL of dry, degassed pyridine was added via a cannula, followed by benzyloxy tetra(ethylene glycol)-G=O—OH (9.57 g, 23.8 mmol) and toluenesulfonylchloride (18.13 g, 95.1 mmol, 4 equiv.) (Acros Organics). The mixture was stirred at RT for 5 days, the solvent removed by rotary evaporation, and the residue taken up in 150 mL of DCM. The organic solution was then poured into 100 mL of 1% (v/v) aqueous HCl, and the organic layer separated using a separation funnel. The aqueous layer was extracted with 50 mL of DCM, and the combined organic fraction was dried over Na₂SO₄. The solution was filtered and the solvent removed to give a clear oil, which crystallizes on standing (18.97 g; 92% yield). An analytical sample was purified by flash chromatography (3:1 EtOAc/hexanes; R_{f}=0.65). Its spectra are as follows:

¹H NMR (CDCl₃): δ 7.71-7.69 (m, 6H), 7.36-7.30 (m, 10H), 7.28-7.25 (m, 1H), 4.56 (2H), 3.90 (6H), 3.68-3.59 (m, 10H), 3.55-3.52 (m, 2H), 3.43-3.40 (m, 2H), 3.36-3.33 (m, 2H), 3.30 (2H), 2.45 (9H); and

¹³C NMR (CDCl₃): δ145.2, 138.2, 131.9, 130.0, 128.2, 128.0, 127.8, 127.6, 127.5, 73.1, 70.7, 70.5, 70.5, 70.4, 69.9, 69.3, 67.2, 66.8, 43.7, 21.6; and

MALDI-TOF MS: C₄₁H₅₂O₁₄S₃; calc. 864.3, found 887.6 [M+Na]⁺ amu.

The following Scheme D-A illustrates this reaction.

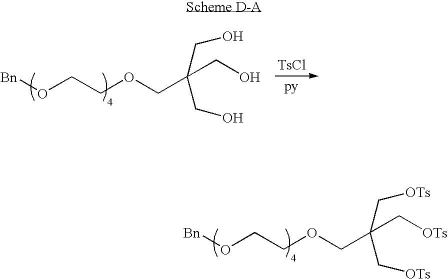

### B. Synthesis of benzyloxy tetra(ethylene glycol)-G=0-Br

Benzyloxy tetra(ethylene glycol)-G=0-OTs (18.0 g, 20.8 mmol) and NaBr (12.85 g, 124.9 mmol, 6 equiv.) (Aldrich) were placed in a 100-mL round-bottom flask. Then 50 mL of DMAc were added and the reaction stirred at 140° C. for 2 hours. The solvent was removed by rotary evaporation and the crude product purified by flash chromatography (1:1 EtOAc:hexanes; R_{f}=0.55) to give a yellow oil (10.16 g; 83% yield). Its spectra are as follows:

¹H NMR (CDCl₃): δ 7.35-7.26 (m, 5H), 4.57 (2H), 3.67-3.63 (m, 16H), 3.54 (m, 8H); and

MALDI-TOF MS: C₂₀H₃₁Br₃O₅; calc. 588.0, found 615.2 [M+Na]⁺ amu.

The following Scheme D-B illustrates this reaction.

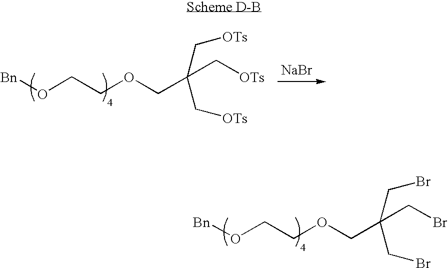

### C. Synthesis of benzyloxy tetra(ethylene glycol)-G=1-propionate

A 100-mL round-bottom flask was plugged with a septum and purged with N₂ gas. Then benzyloxy tetra(ethylene glycol)-G=0-Br (10.20 g, 17.3 mmol) was weighed into the flask and dissolved in 70 mL of dry, degassed DMF. EHTBO (9.30 g, 54.5 mmol, 3×1.05 equiv.) was weighed as a solid into a 100-mL round-bottom flask, purged with N₂ gas, dissolved in 80 mL of dry, degassed DMF, and cannula-transferred into a 500-mL Schlenk flask containing sodium hydride (2.72 g, 64.9 mmol, 3×1.25 equiv.). The reaction was stirred for 2 hours at RT. Then the solution of benzyloxy tetra(ethylene glycol)-G=0-Br was added via a cannula and the mixture heated for 20 hours to 100° C. The solvent was removed by rotary evaporation, the residue dissolved in water and extracted with EtOAc (200 mL) and DCM (2×100 mL). The combined organic extracts were dried over MgSO₄. The solvent was removed by rotary evaporation to give the crude product as yellow oil (17.3 g; 100% yield). Half of the crude product was purified by flash chromatography (EtOAc, R_{f}=0.65) to give benzyloxy tetra(ethylene glycol)-G=1 propionate as light yellow oil (7.15 g; 89% yield). Its spectrum is as follows:

MALDI-TOF MS: C₄₄H₇₆O₂₀; calc. 924.5, found 947.4 [M+Na]⁺ amu.

The following Scheme D-C illustrates this reaction.

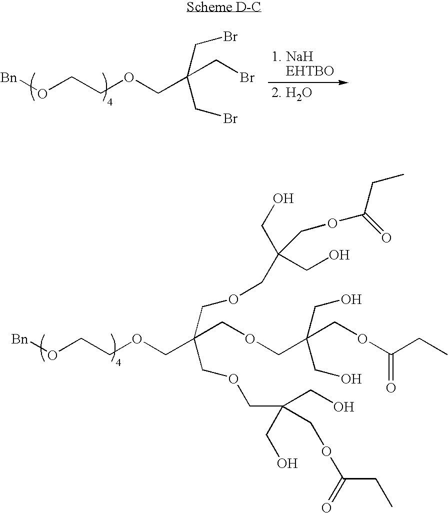

### D. Synthesis of benzyloxy tetra(ethylene glycol)-G=1-OH

Crude benzyloxy tetra(ethylene glycol)-G=1-propionate was dissolved in 100 mL of MeOH. Then 3 mL of concentrated HCl were added and the solution heated to reflux for 1 hour. The reaction was allowed to cool to RT and stirred overnight. The reaction was quenched by adding aqueous NaHCO₃, filtered and dried to give a light yellow oil, which was used without further purification. Its spectrum is as follows:

MALDI-TOF MS: C₃₅H₆₄O₁₇; calc. 756.4, found 779.5 [M+Na]⁺ amu.

The following Scheme D-D illustrates this reaction.

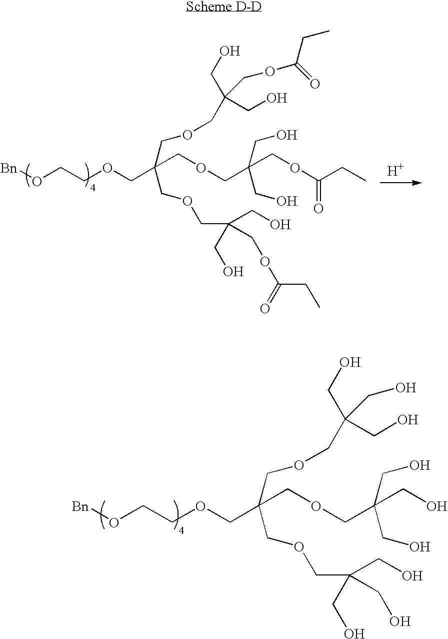

### E. Synthesis of benzyloxy tetra(ethylene glycol)-G=1-OMe

A solution of benzyloxy tetra(ethylene glycol)-G=1-OH (7.0 g) (made from Example D-D and used without purification) in 100 mL of dry, degassed DMF was cannula-transferred into a 500-mL Schlenk flask charged with NaH (6.64 g, 2 equiv.). Vigorous reaction was observed and a gray sponge forms over the course of 10 min. Additional 50-70 mL of DMF was added and the flask shaken to break up the solid. The resulting slurry was stirred for 90 min. at RT. The reaction was cooled to 0° C. in an ice bath, and methyl iodide (13.0 mL, 2.5 equiv) (Aldrich) was slowly added via a syringe. At this point a large amount of gas developed. Gas evolution mostly ceased after 2 hours and the reaction mixture was allowed to stir for 2 days. The reaction mixture was filtered to remove precipitated salts, and the filtrate was dried by rotary evaporation. The solid residue was partitioned between EtOAc and water, extracted with EtOAc (3×100 mL) and the resulting yellow solution dried over Na₂SO₄. The solvent was removed by rotary evaporation and the resulting yellow oil used without further purification. Its spectra are as follows:

¹H NMR (CDCl₃): δ 7.35-7.30 (m, 4H), 7-30-7.25 (m, 1H), 4.56 (br, 2H), 3.69-3.28 (m, 75H); and

¹³C NMR (CDCl₃): δ 138.2, 128.2, 128.0, 127.6, 127.4, 73.1, 72.0, 71.9, 71.4, 71.1, 70.5, 70.4, 70.3, 70.1, 69.8, 69.6, 69.3, 69.1, 59.2, 46.0, 45.4, 45.2, 45.1; and

MALDI-TOF MS: C₄₄H₈₂O₁₇; calc. 882.6, found 905.9 [M+Na]⁺ amu.

The following Scheme D-E illustrates this reaction.

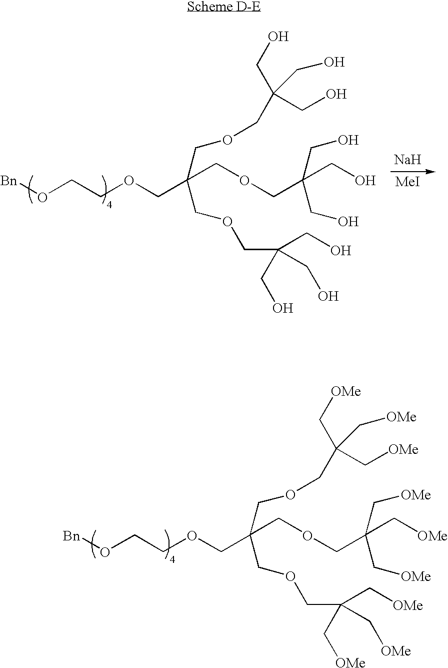

### F. Synthesis of tetra(ethylene glycol)-G=1-OMe

Benzyloxy tetra(ethylene glycol)-G=1-OMe (6.67 g, 6.6 mmol) was dissolved in 50 mL of MeOH in a 200-mL hydrogenation flask. Pd/C (2.0 g, 10% w/w) was added and the bottle was connected to a hydrogenation apparatus overnight at 55 psi. Then the solution was filtered through a pad of Celite to remove the Pd/C catalyst, the filter washed with DCM, and the solvent removed by rotary evaporation. The product was purified by flash chromatography (3:1 EtOAc/acetone; R_{f}=0.65) to give a clear oil (4.62 g; 88% yield). Its spectra are as follows:

¹H NMR (CDCl₃): δ 3.69-3.52 (m, 17H), 3.43-3.29 (m, 58H); and

¹³C NMR (CDCl₃): δ 72.5, 72.0, 71.9, 71.1, 70.5, 70.5, 70.4, 70.3, 70.2, 61.6, 59.3, 46.0, 45.2; and

MALDI-TOF MS: C₃₇H₇₆O₁₇; calc. 792.5, found 815.6 [M+Na]⁺ amu.

The following Scheme D-F illustrates this reaction.

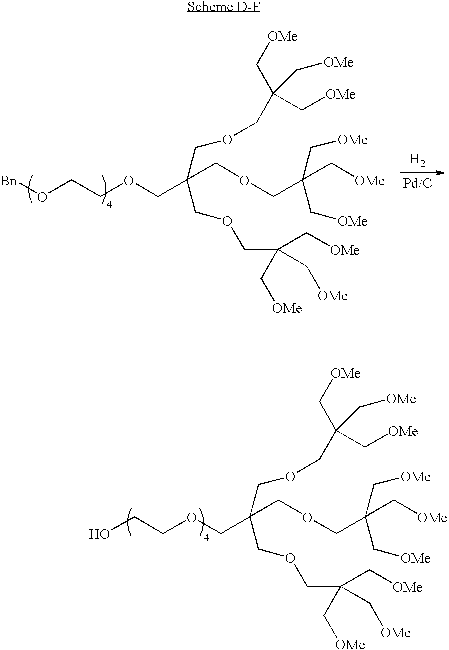

### EXAMPLE E

### A. [Cystamine]; Gen=0; dendri-PAMAM; (acetamide)₄

G=0 PAMAM dendrimer with cystamine core and amine (TF) surface (2.315 g, 3.80 mmol) was dissolved in 5 mL of MeOH. Then TEA (1.847 g, 18.25 mmol) was added to the solution. This mixture was cooled to 0° C. and acetic anhydride (1.725 mL, 18.25 mmol) was added dropwise. The reaction was allowed to warm to RT and stirred overnight. TLC showed that all starting material was consumed. The solvent was removed to give crude product as a brown solid, yielding 3.47 g. 1.27 g of the crude was purified by column chromatography over SiO₂ using a solvent (6:1:0.02 CHCl₃:MeOH:NH₄OH) to give the product as a white solid (593.3 mg): mp 141.0-142.0° C.

¹H NMR (D₂O, 300 MHz): δ ppm 1.82 (s, 12H), 2.25 (m, 8H), 2.64 (m, 16H), 3.19 (t, 16H), 4.67 (s, 8H); ¹³C NMR: 21.92, 32.52, 34.39, 38.60, 38.66, 48.77, 51.43, 174.14, 175.01 ppm.

### B. The reduction of [Cystamine]; Gen=0; dendri-PAMAM; (acetamide)₄ Dendrimer

The dendrimer from Example 8A (148.8 mg, 0.1915 mmol) was dissolved in 2 mL MeOH, which was purged with nitrogen gas for 15 minutes prior to use. DTT (28 mg, 0.182 mmol, 0.95 equiv. per dendrimer) was added to the solution. The reaction mixture was stirred for two days at RT under nitrogen gas. TLC showed that all DTT was consumed, and the product spot was positive to Ellman's reagent on a TLC plate. The product was used in the next reaction without further purification.

### C. Reaction of Focal Point, Thiol Functionalized PAMAM Dendron with Methyl Acrylate

To the reaction solution of Example 8-B was added methyl acrylate (117 mg, 1.36 mmol). The reaction was heated to 40° C. for two hours. TLC showed that there was starting material left. Therefore, another 117 mg of methyl acrylate was added and TLC showed complete reaction after 4 hours. The solvent was removed by rotary evaporation. The residue was purified by column chromatography over SiO₂ to give the product as a pale white solid (104 mg): mp 128.0-129.5° C.

¹H NMR (CDCl₃, 300 MHz): 6 ppm 1.93 (s, 6H), 2.32 (m, 8H), 2.65 (m, 12H), 3.29 (m, 4H), 3.65 (s, 3H); ¹³C NMR: 23.10, 27.13, 29.80, 33.69, 34.58, 39.22, 39.78, 49.86, 51.84, 53.03, 171.27, 172.33, 173.00 ppm.

### D. Reaction of Focal Point, Thiol Functionalized PAMAM Dendron with 2-Isopropenyl Oxazoline

To the reaction solution of Example 8-B was added isopropenyl oxazoline (15.4 mg, 0.136 mmol). The reaction was heated to 40° C. for 2.5 hours. TLC showed that there was starting material left. Therefore another 3.0 mg of isopropenyl oxazoline was added. TLC showed complete reaction after 4 hours. The solvent was removed by rotary evaporation and the residue was purified by column chromatography over siO₂ to give the product as a waxy white solid (58 mg, 85% yield): mp 92.0-95.0° C.

¹H NMR (CDCl₃, 300 MHz): 8 ppm 1.17 (d, J=6.6 Hz, 3H), 1.89 (s, 6H), 2.27 (t, J=6.0 Hz, 6H), 2.47-2.78 (m, 17H), 3.74 (t, J=9.6 Hz, 2H), 4.14 (t, J=9.6 Hz), 7.32 (s, 2H), 7.87 (s, 2H); ¹³C NMR: 17.17, 23.07, 29.98, 33.70, 34.08, 36.11, 39.12, 39.77, 49.91, 52.92, 53.97, 67.37, 170.29, 171.19, 172.99 ppm.

The following Scheme E illustrates this reaction.

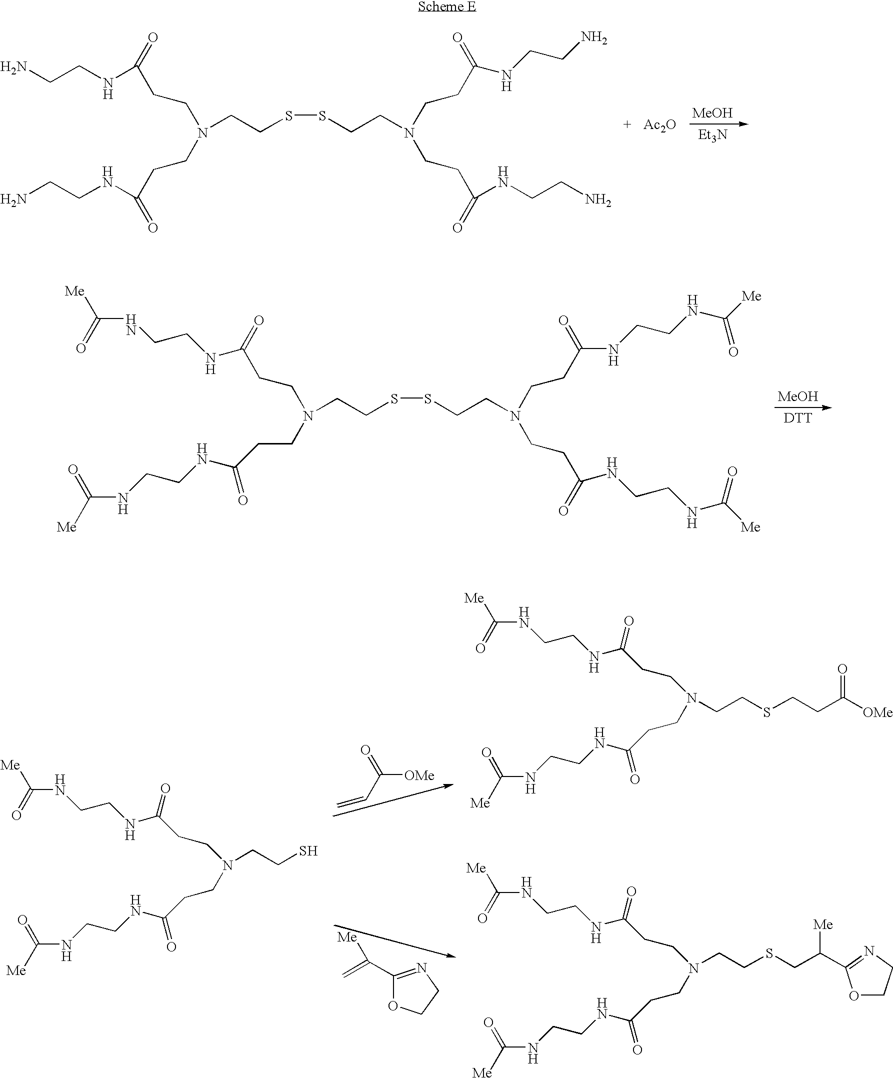

### Syntheses of PAMAM-PERAM Tecto(dendrimers)

### EXAMPLE 1

### Core-Shell Tecto(Dendrimers) with G=4 PAMAM Core and G=1 PEHAM Shell

Core: G=4 PAMAM
Shell: G=1 PEHAM [(C)=TMPTGE; (IF1)=OH; (BR1)=DEIDA; (TF)=Ethyl ester]

To a pressure tube was added a solution of G=1 PEHAM dendrimer with ethyl ester surface (2.17 g, 2.5 mmol, 50 mole equiv. per G=4 PAMAM core; made from Example A) in 11.0 mL of MeOH as the shell unit. To this solution was added lithium chloride (0.21 g, 5.0 mmol, 2 mole equiv. per G=1 ester) (Acros) all at once, and the tube was equipped with a stir bar and stopper. After stirring for 10 mins. at RT, a solution of G=4 STARBURST® PAMAM dendrimer with EDA core and primary amine surface groups (0.71 g, 0.5 mmol, 12.3% w/w solution in MeOH) was added as the core unit, and the tube was closed with a stopper and heated at 45° C. for overnight.

An aliquot of the reaction mixture was analyzed by MALDI-TOF MS and it showed mass peaks at 26,809 (corresponding to approx. 14 G=1 PEHAM dendrimers as the shell) and 54,142 amu (corresponding to approx. 46 G=1 PEHAM dendrimers as the shell). Peaks of low intensities at 80,175 and 106,191 amu indicated the presence of small amounts of cross-linked by-products. Heating was continued for 3 days and progress of the reaction was analyzed by MALDI-TOF MS, showing the same peak intensity ratio.

After 6 days, the reaction mixture was allowed to cool to RT and transferred into a 100-mL, single neck round bottom flask. Then a solution of AEP (2.42 g, 18.75 mmol; 1.25 equiv. per starting G=1 ester group) (Acros) in 10.0 mL of MeOH was added and the mixture heated to 75-80° C. After 22 hours, progress of the reaction was analyzed by IR, revealing the absence of the ester vibration at 1740 cm⁻¹ and the presence of a strong amide vibration band at 1645 cm⁻¹. The MALDI-TOF mass spectroscopy was in good agreement with the conversion of all ester groups into amide functionality. The reaction mixture was allowed to cool to RT, diluted to 2.5-5% w/w solution in MeOH, and subjected to UF, using a 5K size exclusion membrane at a pressure of 15-20 psi (about 135−137.9 kPa) for purification. Its spectra are as follows:

MALDI-TOF (PAMAM-PEHAM tecto(dendrimer) with ester shell surface): 26,809 (PAMAM core with 14 G=1 PEHAM surface dendrimers added) and 54,142 amu (PAMAM core with 46 G=1 PEHAM surface dendrimers added); and

MALDI-TOF (PAMAM-PEHAM tecto(dendrimer) with piperazine shell surface): 37,329 (PAMAM core with 14 G=1 PEHAM surface dendrimers added) and 71,904 amu (PAMAM core with 46 G=1 PEHAM surface dendrimers added).

The following Scheme 1 illustrates this reaction.

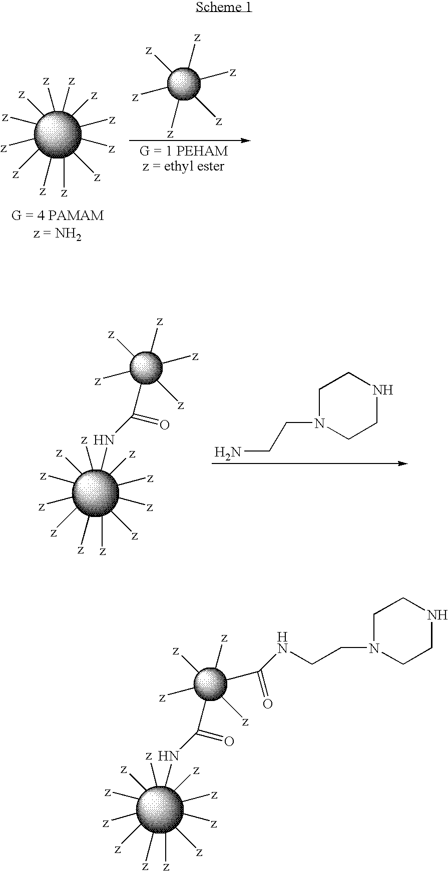

### Syntheses of PEHAM-PEHAM Tecto(dendrimers)

### EXAMPLE 2

### Core-Shell Tecto(Dendrimer) with G=2 PEHAM Core and G=1 PEHAM Shell

Core: G=2 PEHAM [(C)=TMPTGE; (IF1)=OH; (BR1)=DEIDA; (BR2)=TREN; (TF)=Amine]
Shell: G=1 PEHAM [(C)=TMPTGE; (IF1)=OH; (BR1)=DEIDA; (TF)=Ethyl ester]

To an oven dried 100-mL round bottom flask was added G=2 PEHAM dendrimer with primary amine surface (390 mg, 0.265 mmol; made from Example B) dissolved in 4 mL of dry MeOH (Aldrich) as the core unit. The flask was equipped with a stir bar. Then G=1 PEHAM dendrimer with ethyl ester surface (4.6 g, 5.3 mmol, 20 moles equiv. per G=2; made from Example A) dissolved in 11.0 mL of MeOH was added as the shell unit. After stirring for 2 hours at RT, lithium chloride (0.42 g, 10 mmol) (Acros) was added all at once. The reaction flask was arranged with a refluxing condenser and heated at 45° C. overnight under a N₂ atmosphere. Analysis of an aliquot of the sample by MALDI-TOF MS indicated mass peaks for one, two, three, four and five G=1 PEHAM shell units attached to the core, with peak intensities in decreasing order.

Heating was continued for 6 days, then the reaction mixture was allowed to cool to RT. A solution of AEP (5.13 g, 39.75 mmol; 1.25 equiv. per starting G=1 ester) (Acros) in 20 mL of MeOH was added, and the mixture heated to 75-80° C. for 22 hours. Progress of the reaction was monitored by IR revealed the absence of the ester vibration 1740 cm⁻¹ and the presence of a strong amide vibration at 1649 cm⁻¹ after this time period. MALDI-TOF mass spectroscopy supported the complete conversion of ester bonds into amide functionality. The reaction mixture was diluted to 2.5-5% w/w solution in MeOH and subjected to UF using a 3K size exclusion membrane at a pressure of 20-25 psi (about 137.9 kPa) for purification.

MALDI-TOF MS (PEHAM-PEHAM tecto(dendrimer) with ester shell surface): 2349.3, 3232.1, 4011.8 and 4816.8 amu (core unit with 1-4 G=1 shell units added); and

MALDI-TOF MS (PEHAM-PEHAM tecto(dendrimer) with PIPZ shell surface): 2609.4, 3739.7, 4682.3 and 5968.2 amu (core unit with 14 G=1 shell units added).

The following Scheme 2 illustrates this reaction.

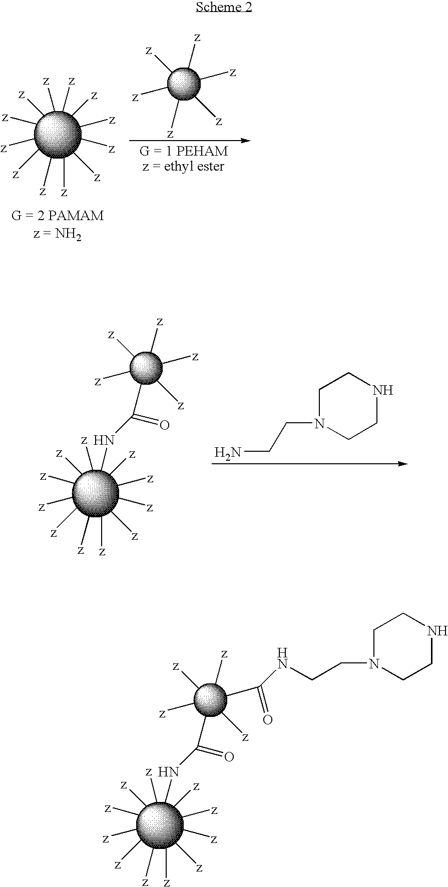

### EXAMPLE 3

### Core-Shell Tecto(Dendrimer) with G=4 PEHAM Core and G=2.5 PEHAM Shell

Core: G=4 PEHAM [(C)=PETGE; (IF1)=OH; (BR1)=PPT; (IF2)=OH; (BR2)=PPT; (IF3)=OH; (BR3)=PPT; (IF4)=OH; (BR4)=TREN; (TF)=Amine]

Shell: G=2.5 PEHAM [(C)=PETGE; (IF1)=OH; (BR1)=PPT; (IF2)=OH; (BR2)=IMDA; (TF)=Ethyl ester]

To a 1.5-dram vial was weighed PEHAM dendrimer G=4, PETGE core, TREN surface (52 mg, 1.4×10⁻³ mmol) and 3 g of MeOH. To a second vial was weighed PEHAM dendrimer G=1, PETGE core, ethyl ester surface (250 mg, 6×10⁻² mmol, 43 equiv. per G=4) and 3 g MeOH. To a third vial was added lithium chloride (62 mg, 1.46 mmol, ˜1 equiv. per ester) and 3 g of MeOH. All three mixtures were made homogeneous and added to a 12-mL glass reaction tube fitted with a pressure relief valve (15 bar, 221 psi) and a stir bar. This mixture was setup in a microwave (Milestone ETHOS MicroSYNTH labstation) with the power set at 400 Watts. This reaction mixture was irradiated with microwaves for 4.9 hours at 50° C. and added dropwise over 5 mins. to TREN (13.0 g, 89.0 mmol, 60 equiv. per ester) in 3 g of MeOH. This mixture was stirred at 25° C. for 67 hours under N₂ gas. An infrared spectrum of this reaction mixture indicated complete disappearance of the ester peak at 1735 cm⁻¹. This mixture was diluted to 300 mL with DI and UF through two 3 KDa cut-off regenerated cellulose membranes to give 600 mL permeate (2 recirculations). With the retentate volume at 150 mL another 1200 mL permeate were obtained (8 recirculations). Volatile material was removed from the retentate by rotary evaporation to give 360 mg crude product. The product was dissolved in 25 mL of DI and UF on a Pellicon XL ultrafiltration device containing 10 KDa cut-off regenerated cellulose membranes to give 250 mL permeate(10 recirculations). Volatile material was removed from the retentate to give 160 mg of purified product. SEC of this product showed low molecular weight material mixed with tectodendrimer product as a bimodal distribution. The retentate was further purified on a Pellicon XL ultrafiltration device containing 30 KDa cut-off regenerated cellulose membranes in 15 mL of DI to give 150 mL permeate (10 recirculations). Volatile material was removed from the retentate by rotary evaporation to give 90 mg product.

The following Scheme 3 illustrates this reaction.

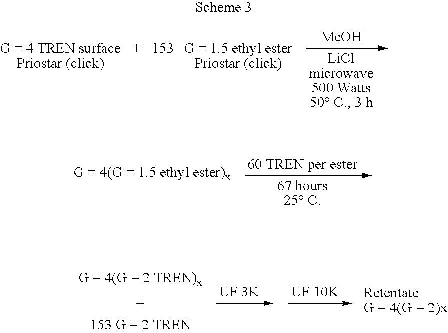

SEC analysis: Symmetrical peak between 16.0 and 20.0 mins. elution time with maximum at 18.0 min. (M_{z}/M_{w}=1.5).

### EXAMPLE 4

### Core-Shell Tecto(Dendrimer) with G=4 PEHAM Core and G=1.5 PEHAM Shell

Core: G=4 PEHAM [(C)=PETGE; (IF1)=OH; (BR1)=PPT; (IF2)=OH; (BR2)=PPT; (IF3)=OH; (BR3)=PPT; (IF4)=OH; (BR4)=TREN; (TF)=Amine]
Shell: G=1.5 PEHAM [(C)=PETGE; (IF1)=OH; (BR1)=IMDA; (TF)=Ethyl ester]

To a 1.5-dram vial was weighed PEHAM dendrimer G=4, PETGE core, TREN surface (55.0 mg, 1.5×10⁻³ mmol) and 3 g of MeOH. To a second vial was weighed PEHAM dendrimer G=1.5, PETGE core, ethyl ester surface (257.0 mg, 2.3×10⁻¹ mmol, 153 equiv. per G=4) and 3 g of MeOH. To a third vial was added lithium chloride (99.0 mg, 23.0 mmol, ˜12 equiv. per ester) and 3 g of MeOH. All three mixtures were made homogeneous and added to a 12-mL glass reaction tube fitted with a pressure relief valve (15 bar, 221 psi) and a stir bar. This mixture was setup in a microwave (Milestone ETHOS MicroSYNTH labstation) with the power set at 400 Watts. This reaction mixture was irradiated with microwaves for 4.9 hours at 50° C. and added dropwise over ˜5 mins. to TREN (16.0 g, 110.0 mmol, 60 equiv. per ester) in 3 g of MeOH. This mixture was stirred at 25° C. for 67 hours under N₂ gas. An IR of this reaction mixture indicated complete disappearance of the ester peak at 1735 cm⁻¹. This mixture was diluted to 300 mL with DI and UF on two 3 KDa cut-off regenerated cellulose membranes to give 600 mL permeate (2 recirculations). With the retentate volume at 150 mL another 1200 mL permeate were obtained (8 recirculations). Volatile material was removed by rotary evaporation to give 160 mg crude product. SEC of this product showed some low molecular weight material mixed with tectodendrimer product as a bimodal distribution, containing some residual TREN and unreacted shell reagent. This mixture was dissolved in 25 mL of DI and UF on a Pellicon XL ultrafiltration device containing 10 KDa cut-off regenerated cellulose membranes to give 250 mL permeate (10 recirculations). MALDI-TOF mass spectrum analysis of this material indicated a broad peak at 46 kDa.

The following Scheme 4 illustrates this reaction.

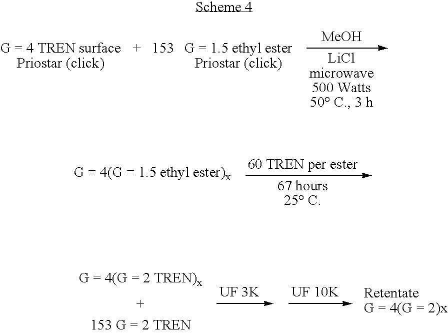

SEC analysis: Bimodal distribution with some low molecular weight material mixed with tectodendrimer product.

### Syntheses of PAMAM-PAMAM Tecto(dendrimers)

### EXAMPLE 5

### Core-Shell Tecto(Dendrimer) with G=6 PAMAM Core and G=3.5 PAMAM Shell

Core: G=6 PAMAM [(C)=EDA; (TF)=Amine]
Shell: G=3.5 PAMAM [(C)=EDA; (TF)=Methyl ester]
   A. To an oven dried 500-mL round bottom flask was added G=3.5 PAMAM dendrimer with methyl ester surface (32 g) dissolved in 32 g of dry MeOH (Aldrich) as the shell units. The flask was equipped with a stir bar. To this mixture was added lithium chloride (7 g, Acros). The mixture was stirred until homogenous. Then a mixture containing G=6 PAMAM dendrimer with primary amine surface (6 g) dissolved in 20 g of MeOH as the core unit was added dropwise over 10 mins. The mixture was warmed to 25° C. and placed in a constant temperature bath at 40° C. for 25 days. The core-shell tectodendrimers had methyl ester terminal groups.

After 25 days at 40° C., the mixture was cooled to RT and TRIS (42 g) and potassium carbonate (22 g) was added. The resulting mixture was vigorously stirred for 18 hours at RT. The mixture was purified in DI water using an Amicon stainless steel tangential flow UF having 30 KDa cut-off regenerated cellulose membrane to give 6 L of permeate and 800 mL of UF retentate. The retentate was filtered through a Whatman No. 1 filter paper, freed of volatiles on a rotary evaporator, and evacuated with a high vacuum at 25° C. to give the desired product (20 g).

B. When Part A was repeated using the following substitutions, the desired indicated core-shell tecto(dendrimers) were obtained.

| Example | Core | Shell |
|---|---|---|
| 5B1 | G = 4 | G = 3.5 |
| | [(C) = EDA; (TF) = Amine] | [(C) = EDA; (TF) = Methyl |
| | | ester] |
| 5B2 | G = 5 | G = 2.5 |
| | [(C) = EDA; (TF) = Amine] | [(C) = EDA; (TF) = Methyl |
| | | ester] |
| 5B3 | G = 7 | G = 4.5 |
| | [(C) = EDA; (TF) = Amine] | [(C) = EDA; (TF) = Methyl |
| | | ester] |

This Example 5 is derived from the process of U.S. Pat. No. 6,635,720.

### EXAMPLE 6

### Core-Shell Tecto(Dendrimer) with G=5 PAMAM Core and G=2.5 PAMAM Shell

Core: G=5 PAMAM [(C)=DAB; (TF)=Amine]
Shell: G=2.5 PAMAM [(C)=DAB; (TF)=Methyl ester]
   A. To a 100-mL round bottom flask containing a large stir bar and fitted with a N₂ gas bubbler was added PAMAM dendrimer, DAB core, G=2.5 methyl ester surface (8 g, 1.33 mmol, 31 equiv. per G=5) and 10 g of MeOH. To this homogeneous solution was added lithium chloride (2.0 g, 47 mmol, ˜1 equiv. per methyl ester) under stirring. This solution was cooled to 4° C., then PAMAM dendrimer, DAB core, G=5, amine surface (1.2 g, 4.15×10⁻² mmol) dissolved in 5 g of MeOH was added dropwise over 2-3 min. The resulting mixture was warmed to 40° C., sealed with a polypropylene cap and Parafilm, and kept at 40° C. in an oil bath for 25 days.

Then the mixture was diluted with 100 mL of MeOH and added to a dropping funnel attached to a 500-mL round bottom flask containing a large stir bar, TREN (250 g, 1.71 mol, 41 equiv. per ester), and 20 g of MeOH, cooled to 4° C. The reaction mixture was added dropwise to the well-stirred amine solution over 2 hours. The mixture was allowed to warm to 25° C. and stirred under N₂ gas for 3 days. Complete reaction was monitored by the disappearance of the ester peak at 1735 cm⁻¹ in IR. The mixture was split in half for purification on tangential flow UF containing one 30 KDa cut-off membrane. Each half of the mixture weighed 175 g and was diluted to 4 L with DI to give 4-5% solids (w/w). After 4 L of permeate were obtained, the mixture was concentrated to 2 L retentate volume and 2 L of permeate was collected. This retentate was concentrated to 1 L and 1 L of permeate was collected. This retentate was concentrated to 500 mL and 3 L of permeate were collected. This mixture was removed from the UF and the UF washed with 3×100 mL DI. Combined washes and retentate were stripped of volatiles by rotary evaporation to give a viscous, colorless residue, which was dissolved in 100 mL of MeOH and stripped of volatiles by rotary evaporation four times. The residue was then dried to constant weight at high vacuum to give 1.5 g of product. The second aliquot was worked up the same way to give 1.7 g of product for a combined total of 3.2 g for the G=5(G=3 TREN) core-shell tecto(dendrimer) product.

B. When Part A was repeated using the following substitutions, the desired indicated core-shell tecto(dendrimers) were obtained.

| Example | Core | Shell |
|---|---|---|
| 6B1 | G = 6 | G = 2.5 |
| | [(C) = DAB; (TF) = Amine] | [(C) = DAB; (TF) = Methyl |
| | | ester] |
| 5B2 | G = 6 | G = 3.5 |
| | [(C) = DAB; (TF) = Amine] | [(C) = DAB; (TF) = Methyl |
| | | ester] |

### EXAMPLE 7

### Core-Shell Tecto(Dendrimer) with G=3.5 PAMAM Core and G=2 PAMAM Shell

Core: G=3.5 PAMAM [(C)=DAB; (TF)=Methyl ester]
Shell: G=2 PAMAM [(C)=DAB; (TF)=Amine]
   A. To a 100-mL round bottom flask containing a large stir bar and fitted with a N₂ gas bubbler was added PAMAM dendrimer, DAB core, G=2, amine surface (8.0 g, 2.56 mmol, 25 equiv. per G=3.5) and 30 g of MeOH. To this homogeneous solution was added lithium chloride (300 mg, 7.0 mmol, ˜1 equiv. per methyl ester) under stirring. The solution was cooled to 4° C., then PAMAM dendrimer, DAB core, G=3.5, methyl ester surface (1.2 g, 0.1 mmol) dissolved in 5 g of MeOH was added dropwise over 2-3 min. The resulting mixture was warmed to 40° C., sealed with a polypropylene cap and Parafilm, and stirred in an oil bath at 40° C. for 25 days.

The mixture was diluted with 100 mL of MeOH and added to a dropping funnel attached to a 500-mL round bottom flask containing a large stir bar, EA (2.0 g, 33.0 mmol, 6 equiv. per ester) and 20 g of MeOH, cooled to 4° C. The reaction mixture was added to the well stirred amine solution over 2 hours. This mixture was allowed to warm to 25° C. and stirred under N₂ gas for 3 days. Complete reaction was monitored by the disappearance of the ester peak at 1735 cm⁻¹ in IR. Then the mixture was diluted to 250 ml with DI and purified on tangential flow UF containing one 10 KDa cut-off membrane. After 2.5 L of permeate were obtained, this mixture was removed from the UF, and the UF washed with 3×100 mL DI. combined washes and retentate were stripped of volatiles by rotary evaporation to give a viscous, colorless residue. This residue was dissolved in 100 mL of MeOH and stripped of volatiles by rotary evaporation four times. This residue was dried to constant weight at high vacuum to give 3.2 g of G=4EA(G=2) core-shell tecto(dendrimer) product.

B. When Part A was repeated using the following substitutions, the desired indicated core-shell tecto(dendrimers) were obtained.

| Example | Core | Shell |
|---|---|---|
| 7B1 | G = 3.5 | G = 3 |
| | [(C) = DAB; (TF) = Methyl ester] | [(C) = DAB; (TF) = |
| | | Amine] |
| 7B2 | G = 5.5 | G = 3 |
| | [(C) = DAB; (TF) = Methyl ester] | [(C) = DAB; (TF) = |
| | | Amine] |

### Encapsulation Efficiency of Tecto(dendrimers) for Indomethacin

### EXAMPLE 8

### PAMAM-PEHAM and PEHAM-PEHAM Tecto(Dendrimers) from Examples 1 and 2, Respectively, were Tested for their Encapsulation Efficiency in Di Water, Using the Drug Indomethacin

### Method

Encapsulation efficiency of indomethacin was examined in the presence of tecto(dendrimers) (˜0.2% w/v) in 5 mL of DI water. An excess (˜15 mg) of indomethacin (Alfa Aesar) was added to vials containing aqueous dendrimer solutions. These suspensions were briefly sonicated, incubated overnight at 37° C. and shaking (100 rpm) in a shaking water bath, then allowed to equilibrate at RT. The suspensions were filtered through a 0.2 μm pore size nylon syringe filter (13 mm in diameter) (Fisher Scientific) to remove excess drug. PAMAM-PEHAM tecto(dendrimers) were clogging the 0.2 μm filter pores, and therefore, these samples were centrifuged at 4000 rpm for 15 mins. and then filtered through 0.2 μm nylon filter. Samples were analyzed for dendrimer-encapsulated indomethacin by UV spectroscopy at 320 nm on a Perkin Elmer Lambda 2 UV/VIS Spectrometer.

### Results

| | | Molecular | Mole ratio |
|---|---|---|---|
| | | weight | [mole drug/mole |
| Tecto(dendrimer) | Example | [Da] | dendrimer] |
| PEHAM-PEHAM | 2 | 2609 | 5.6 ± 0.121 |
| PAMAM-PEHAM | 1 | 37329 | 46.05 ± 4.501 |

### Syntheses of Dendronized Dendrimers

### EXAMPLE 9

### Polyether Dendron G=1 with Methoxy (TF) Attached to PAMAM G=2 Core

Core: G=2 PAMAM with EDA core and primary amine surface
Shell: G=1 Polyether [(C)=Pentaerythritol; (FF)=OH; (EX1)=Succinic ester; (EX2)=Tetra(ethylene glycol); (BR)=Pentaerythritol; (TF)=OMe]

### A. Synthesis of tetra(ethylene glycol)-G=1-OMe succinic ester

To a solution of tetra(ethylene glycol)-G=1-OMe (4.62 g, 5.80 mmol) in 25 mL of pyridine was added succinic anhydride (6.0 g, 58.0 mmol, 10 equiv.) and the resulting solution stirred at 40° C. for overnight. The solvent was removed by rotary evaporation, the solid residue redissolved in 100 mL of water and the solvent removed again. The crude product was dissolved in water, the pH adjusted to 2.0 using HCl, and the solution extracted with DCM (1×150 mL, 2×100 mL). TLC (EtOAc) analysis confirmed the complete removal of succinic acid by-product. The combined extracts were dried over Na₂SO₄ and the solvent was removed by rotary evaporation to give the product as clear oil (5.30 g; 99% yield). Its spectra are as follows:

¹H NMR (CDCl₃): δ 4.27-4.24 (m, 2H), 3.71-3.54 (m, 16H), 3.45-3.27 (m, 57H), 2.65-2.61 (m, 4H); and

¹³C NMR (CDCl₃): δ 172.3, 72.0, 71.9, 71.1, 70.5, 70.4, 70.3, 70.2, 70.1, 69.0, 63.6, 59.3, 46.0, 45.2, 29.5, 29.3; and

MALDI-TOF MS: C₄₁H₈₀O₂₀; calc. 892.5, found 915.8 [M+Na]⁺ amu.

The following Scheme 5A illustrates this reaction.

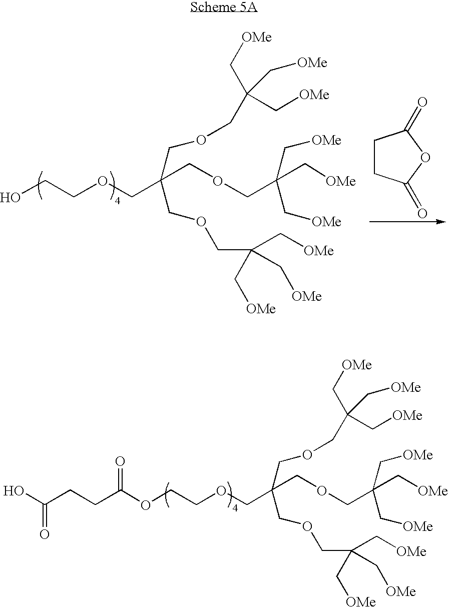

B. Conjugation between PAMAM G=2 core and polyether tetra(ethylene glycol)-G=1-OMe succinic ester shell.

G=2 PAMAM dendrimer with EDA core and NH₂ surface (21.0 mg, 6.1×10⁻³ mmol) was dissolved in 8 mL of DI water. Then tetra(ethylene glycol)-G=1-OMe succinic ester G=1 dendron (185.0 mg, 0.20 mmol, 2 equiv.) was added and the solution stirred for 5 min. DCC (43.0 mg, 0.20 mmol, 2 equiv.) (Aldrich) was added as a solid, and the slurry was allowed to stir overnight at RT. A sample for MALDI-TOF MS was prepared and the reaction mixture dried by rotary evaporation. The crude solid was resuspended in a small amount of water, and solid material separated by centrifugation. The solution was decanted and dialyzed in water (1 kDa dialysis membrane, 18-mm diameter, 12-cm in length, Spectra/Por®, Spectrum Laboratories). The final product was isolated by lyophilization as clear wax (104 mg; 91% yield). Its spectrum is as follows:

MALDI-TOF MS: 15,401 amu [PAMAM core with 14 G=1 dendrons added], 16,487 amu [PAMAM core with 15 G=1 dendrons added] and 17,137 amu [PAMAM core with 16 G=1 PEHAM surface dendrons added).

The following Scheme SB illustrates this reaction.

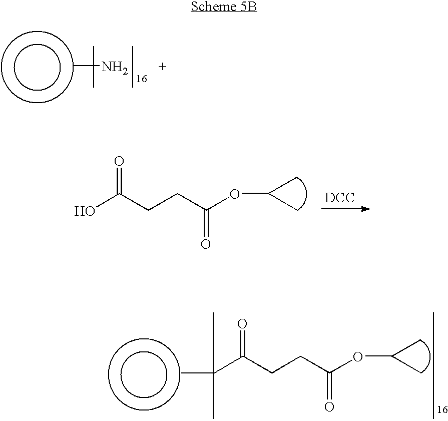

### EXAMPLE 10

### Encapsulation of Indomethacin into Dendronized Dendrimer (PAMAM G=2 Core with Polyether G=1 shell

80 mg dendronized dendrimer from Example 9 were dissolved in 8 mL of a 62.5:37.5 water-MeOH (% v/v) mixture. A 1-mL aliquot (in duplicate) from this stock solution was added to 4 mL water (0.2% w/v). Indomethacin powder (10.0 mg) was added to the dendrimer solution, briefly sonicated, and kept overnight in a shaking water bath at 37° C. and 100 rpm. The suspension was filtered through a 0.2 μm nylon filter. The indomethacin content of the filtrate was measured using UV light at 320 nm. As a control, indomethacin was dissolved in a dendrimer-free solvent mixture (62.5:37.5 water-MeOH, % v/v). The results were compared to the encapsulation efficiency of PAMAM dendrimers of different generations and surfaces. The indomethacin encapsulation efficiency of the G=2 core/G=1 shell dendronized dendrimer was comparable to G=3/G=4 PAMAM dendrimers. The results are shown in the Table below.

| PAMAM EDA (generation - | Indomethacin loading |
|---|---|
| surface) | [molecules/dendrimer] |
| PAMAM G = 2 + EO G = 1 OMe | 3.7 (±0.24 SD) |
| G = 3 - NH₂ | 4.2 (±0.14 SD) |
| G = 4 - NH₂ | 11.7 (±0.89 SD) |
| G = 3 - PEG | 3.0 (±0.18 SD) |
| G = 4 - PEG | 6.6 (±0.26 SD) |
| G = 3 - TRIS | 2.9 (±0.02 SD) |
| G = 4 - TRIS | 5.3 (±0.26 SD) |
| G- 3 - pyrrolidone | 2.8 (±0.03 SD) |
| G = 4 - pyrrolidone | 5.8 (±0.16 SD) |
| G- 3 - succinic acid | 4.2 (±0.14 SD) |
| G = 4 - succinic acid | 4.2 (±0.14 SD) |

The advantage of using these dendronized dendrimers is they are more quickly made with greater purity than the PAMAM counterpart G=3 and G=4 dendrimers. Thus these dendronized dendrimers have commercial advantages while performing comparably. Syntheses of Core-Shell tecto(dendrimers) where [C] and/or [S] contain a cleavable bond

### EXAMPLE 11

G=1 PAMAM dendrimer with cystamine core and amine (TF) as the [C] (232 mg, 0.152 mmol) and G=1 PAMAM dendrimer with cystamine core and carboxylic acid (TF) as the [S] (180 mg, 0.076 mmol) were dissolved in 8 mL of DI water. Then LiCl (100 mg, 2.36 mmol) was added and the mixture was stirred at RT for 36 hours. Then 1-[3-(dimethylamino)propyl]-3-ethylcarbodiimide hydrochloride (409 mg, 1.88 mmol) was added and the reaction was stirred for 24 hours. The reaction was dialyzed through a 1K regenerated cellulose membrane against DI water. Water was removed and the residue was purified by UF through 30K, 10K, 5K Pellicon membranes. The permeate and retentate of each filtration was analyzed by PAGE.

According to the PAGE results, the shape and yield of tecto(dendrimers) could be determined. G=1 dendrimers with —S—S— core components were found to form linear tecto(dendrimers) as shown in Scheme 6A below.

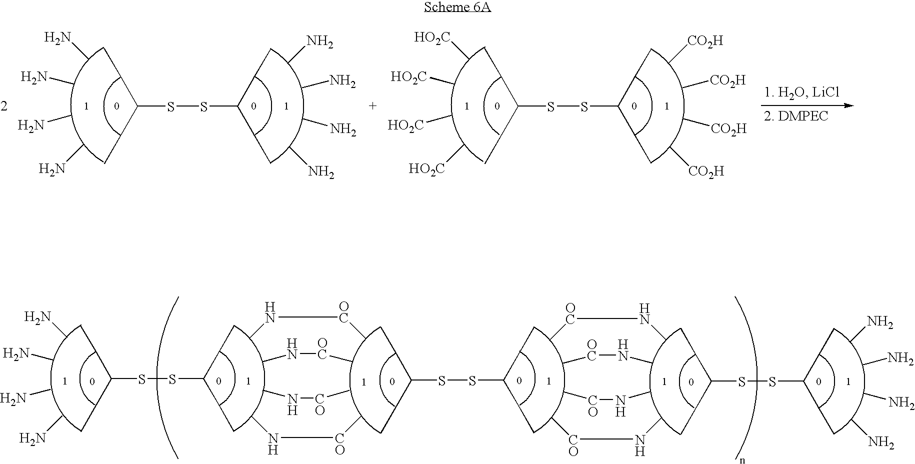

The PAGE of the crude product indicated there is a main product with a molecular weight of about 120,000 Dalton, and the corresponding polymerization number is 64. In addition a minor product of partially branched 64-mer was found. The results of PAGE are summarized below.

| | Partially | | |
|---|---|---|---|
| Linear polymer | Branched | Unreacted | Loss during |
| (64-mer) | (64-mer) | material | filtration |
| 38% | 12% | 32% | 18% |

Using the exactly same conditions, G=1 PAMAM dendrimers [C] with hexyldiamine core moiety and amino (TF) and G=1 PAMAM dendrimers [S] with hexyldiamine core moiety and carboxylic acid (TF) were polymerized (Scheme 6B). These hexyldiamine dendrimers have the exact same number of atoms in the core as the cystamine dendrimers. Therefore, they should act the same way if there is no core-related property differences. However, the PAGE result of the reaction showed that hexyldiamine core dendrimers only formed 8-mer rather than 64-mer.

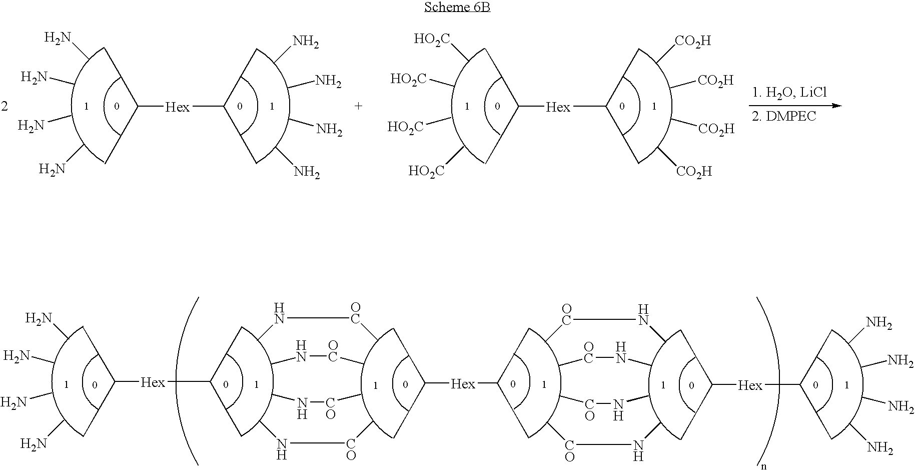

The different results between these two dendrimeric polymerizations above must occur because of the core differences. Prior studies have shown that the lone pair protons of the sulfur within cystamine play an important role during polymerization, causing strong hydrogen bonds between different dendrimers, resulting in dimer formation. The same mechanism is believed to be responsible for the formation of longer polymer chains formed from cystamine core dendrimers compared to hexyldiamine core dendrimers.

Using the exactly same conditions, G=2 PAMAM dendrimers [C] with cystamine core moiety and amino (TF) and G=2 PAMAM dendrimers [S] with cystamine core moiety and carboxylic acid (TF) were polymerized in a 1:2 ratio (Scheme 6C). PAGE results for this reaction indicate the formation of polymers with higher branching dispersity, such as hyperbranched or spherical tecto(dendrimers) as shown below.

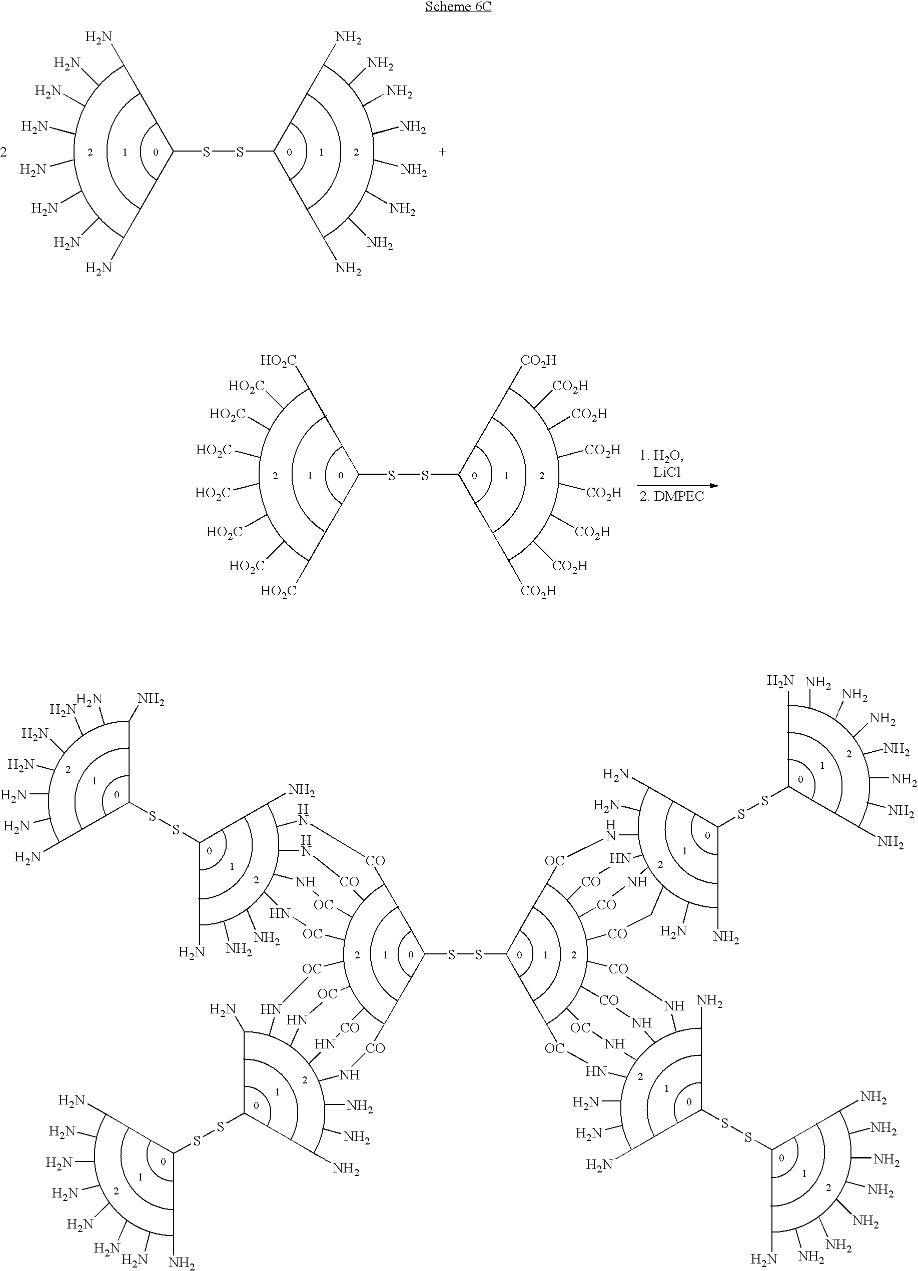

### EXAMPLE I

### Core/Shell Results from Quantigene Assay

### A. Transfection

MDCK cells and HEK cells were seeded to achieve ˜70% confluency in a 48 well tissue culture dish (Becton Dickinson). Transfection reagents: Lipofectamine™ 2000 (Invitrogen), G=4EA(G=2) core-shell tecto(dendrimer) (450 μg/mL) and G=5(G=3TREN) core-shell tecto(dendrimer) (200 μg/mL) were diluted using complete MEM, with the exception of Lipofectamine™ 2000 which was in media lacking FBS and antibiotics to the desired concentration. At the same time siRNA for Cyclophilin B (Dharmacon) or non-targeting siRNA (siCONTROL™ Non-Targeting siRNA #2, Dharmacon) was diluted in media to a concentration to achieve 150 nM in the final transfection. The transfection agents and siRNA mixtures were incubated at RT separately for 15 mins. Equal volumes (125 μL each) of transfection agent and siRNA were mixed together and incubated for 20 mins. to form transfection complexes. Media was removed from the cells and transfection mixtures added. Cells were then incubated at 37° C. in 5% CO₂. Cell culture media was changed to fresh complete MEM for all samples at 6 hours post-transfection. Cells were again incubated at 37° C., 5% CO₂ until harvested for the bDNA assay at 48 hours post-transfection.

B. bDNA Assay

To harvest the cells for the bDNA assay 125 μL (50% volume of media) of Lysis mixture (Genospectra) was added to each well. Cells were observed under the phase contrast microscope to ensure complete lysis. Cell lysates were transferred to microcentrifuge tubes and frozen at −20° C. until used for the assay.

Probe set stocks for both Cyclophilin B (PPIB, Genospectra) and β-actin (ACTB, Genospectra) (as a non-targeted control) were prepared as per the QuantiGene™ protocol by mixing 52 μL of the 5× probe solutions (CE, LE, and BL) with 208 μL of TE (10 mM TRIS, 1 mM ethylenediaminetetraacetate) and frozen at −20° C. Probes for detection were prepared by mixing 1.44 mL of Lysis mixture, 2.87 mL of water, and 80 μL of each probe set component (CE, LE, BL). In each well of a 96 well capture plate (Genospectra), 65 μL of the probe solution was mixed with 35 μL (˜10,000 cell equivs.) of cell lysate from the transfection. The capture plate was sealed and incubated at 50° C. overnight.

After 16 hours incubation, 250 μL of wash buffer (1×SSC [0.15 M NaCl, 0.015 M sodium citrate], 0.1% lithium laurylsulfate) was added to each well to wash and the solution was poured off. The wells were rinsed 3 times with 250 μL wash buffer and the plate dried by inverting and tapping on a paper towel. To each well 100 μL of amplification solution (Genospectra) was added and the plate incubated at 50° C. for 1 hour. The amplification solution was poured off and the wells washed and dried as above. To each well was then added 100 μL of label solution (Genospecta) and the plate incubated at 50° C. for 1 hour. The label solution was then poured off and the wells washed and dried as above. To each well was then added 100 μL of substrate (Genospectra) and incubated at 50° C. for 15 mins. The luminescence was then detected on a GloRunner™ (Turner Biosystems) multiwell plate reading luminometer using the default software settings. Average values and standard deviations for the repeat transfections were calculated.

The luminescence for the targeted gene, PPIB, was adjusted to account for variability in total RNA in the lysates by dividing the measured value by an adjustment factor that was calculated by dividing the measured ACTB signal by the control (mock transfection) signal:

adjusted PPIB=measured PPIB/(measured ACTB/control ACTB) Formula A

The percent knockdown relative to the control was then calculated by dividing the adjusted PPIB by the control PPIB, multiplying by 100 to give a relative percent expression; this is then subtracted from 100 to give percent knockdown:

percent knockdown=100−(100*(adjusted PPIB/control PPIB)) Formula 2

### C. Results/Conclusions:

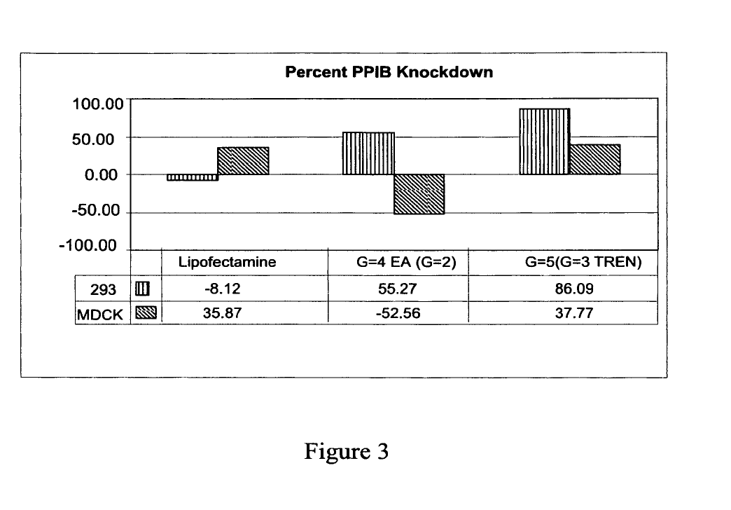

The results of these calculations are shown in FIG. 3. In the HEK 293 cell line the G=4EA(G=2) (55.27%), and G=5(G=3TREN) (86.09%) both showed significant gene knockdown of Cyclophilin B (PPIB) at the concentration used. In the MDCK cell line only the G=5(G=3TREN) (37.77%) showed knockdown of the target gene, Cyclophilin B at the concentration used. All of these values are greater than that seen for Lipofectamine™ 2000, a commonly used, commercially successful transfection agent. These results show that these core-shell tecto(dendrimers) of Formula I can work as highly effective siRNA transfection agents.

Some of the transfection agents, Lipofectamine (−8.12%) in HEK 293 cells and

G=4EA(G2) (−52.56%) in MDCK cells showed results with negative knockdown numbers. This occurs when the knockdown for the ACTB relative to the control is greater than the knockdown of the PPIB relative to the control. Due to the adjustment of PPIB levels based on normalization of ACTB levels (Formula A) this leads to an apparent induction of PPIB even if the unadjusted PPIB reading is lower than the control. This situation can occur for two reasons: toxicity of the transfection agent at the concentration used or non-specific knockdown leading to the decrease in expression of both ACTB and PPIB. Neither of these causes is desirable for a transfection agent and indicates that the construct does not work well as a transfection agent using the specific conditions tested (it may work well under different conditions).

### EXAMPLE II

### siTox Protocol

### A. Cell Culture

MDCK cells and HEK cells in MEM+10% FBS (complete media) were seeded into 96-well tissue culture plates (Becton Dickinson) at ˜70% confluency, in 100 μL media. The cells were incubated overnight at 37° C., 5% CO₂.

### B. Transfections

Prior to transfections the following stocks were prepared and stored frozen at −20° C.:

1) siCONTROL™ Tox (siTox, Dharmacon) was prepared by dissolving 20 nmol in 4 mL 1× siRNA Buffer (800 μL 5× siRNA Buffer [Dharmacon]+3.2 mL RNase-free sterile water).

2) siCONTROL Non-Targeting siRNA #2 (ns, Dharmacon) was prepared by dissolving 10 nmol in 200 μL 1× siRNA Buffer.

3) A 100 mg/mL stock of dendrimer sample was prepared by filtering a dendrimer solution through a 0.2 μm PVDF syringe filter (Whatman), drying the sample on a lyophilizer, and resuspending at 100 mg/mL in RNase-free sterile water.

The siTox siRNA for each experiment was prepared by adding 2 μL to 48 μL complete media for each well to be transfected with siTox. The ns siRNA for each experiment was prepared by adding 0.2 μL to 49.8 μL complete media for each well to be transfected with ns. The 100 mg/mL dendrimer stock solution was diluted with complete media to 1 mg/mL to create a working solution. Fifty microliters of dendrimer were prepared for each well to be transfected by diluting the working solution to twice the final desired concentration in complete media. The solutions were then incubated for 15 mins. at RT.

Following this incubation, 50 μL of diluted dendrimer (or complete media for control transfection) was mixed with 50 μL of the appropriate siRNA (or complete media for mock transfection). The samples were then incubated for 20 mins. at RT to form transfection complexes. Media was aspirated from the cell cultures, and 100 mL of transfection mixture was added to each well. The cells were incubated with the transfection complexes at 37° C., 5% CO₂ for 6 hours before the media was aspirated again and replaced with 100 μL of complete media. After this step, the cells were incubated at 37° C., 5% CO₂ until assayed for cell survival 48 hours post-transfection.

### C. Transfection Efficiency Assay

A 5 mg/mL solution of MTT (Aldrich) in 1×PBS (0.02 M phosphate, 0.15 M NaCl) pH 7.4 was prepared. Of this solution, 20 μL was added to each well of the 96 well plate and incubated at 37° C., 5% CO₂ for 5 hours. The media in each well was then aspirated and 200 μL of DMSO (Acros) added to each well and incubated 5 mins. at 37° C., 5% CO₂. The absorbance of each well was then measured at 570 nm and 690 nm on a ThermoLabsystems™ Multiskan MCC/340 microplate reader to analyze the transfection efficiency. After subtracting the 690 nm from the 570 nm reading to remove background, the percent survival rate was calculated using the formula:

Percent survival=100*(sample reading/relevant control reading). Formula C

D. Results and conclusions

The core-shell tecto(dendrimers) of Formula I tested were: G=6(G=3TRIS) made in Example 5; G=5(G=3TREN) made in Example 6; G=6(G=3TREN) made in Example 6B1; G=6(G=4TREN) made in Example 6B2; G=4EA(G=2) made in Example 7; G=4EA(G=3) made in Example 7B1; and G=6EA(G=3) made in Example 7B2.

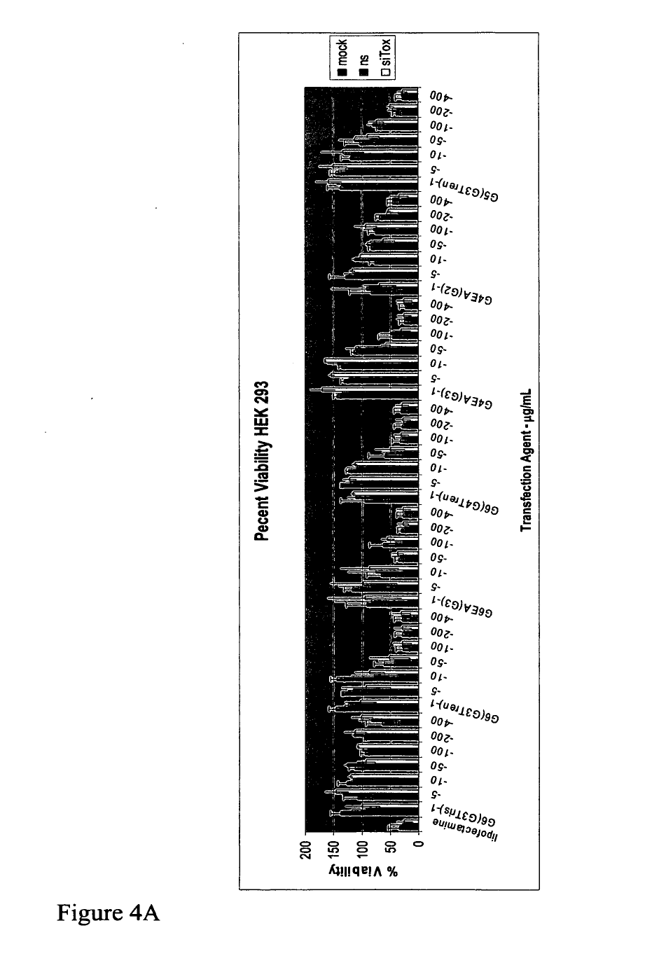

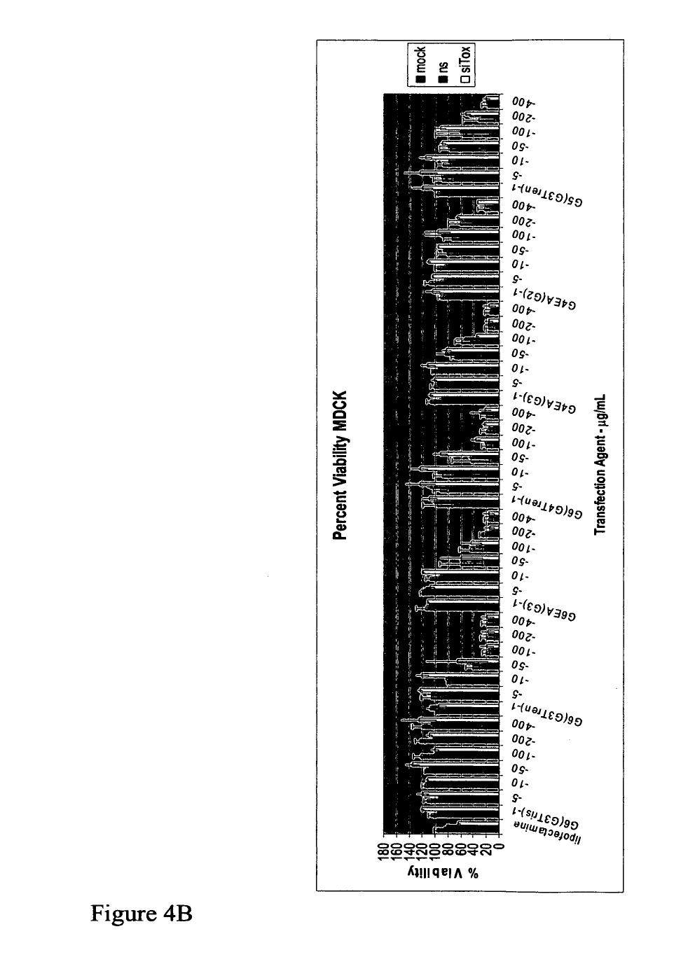

Shown in FIGS. 4A and B are the average results of two transfection experiments with standard deviations in HEK 293 cells and MDCK cells. Transfections were performed with a range of concentrations of each dendrimer from 1 to 400 μg/mL (1, 5, 10, 50, 100, 200, 400 μg/mL).

The siTox siRNA induces cell death by apoptosis upon successful transfection. Therefore a decrease in viability when siRNA is transfected is the desired result. This can be visualized in the above graphs in the sets of three bars for each test concentration by a right bar (yellow) being shorter than the two left bars (blue and red, mock and ns, respectively). If both right bars (red and yellow, ns and siTox, respectively) are both shorter than the left (blue, mock) it indicates non-specific knockdown leading to cell death. Lastly, if all three are very low it indicates toxicity leading to cell death caused by the transfection agent.

In HEK 293 cells, Lipofectamine had a fairly high toxicity and some knockdown as the siTox was slightly lower than the controls. The G=6(G=3TRIS) showed some non-specific knockdown at 1 μg/mL and possibly slight specific knockdown at 50 μg/mL and possibly a little toxicity at the highest concentration used, 400 μg/mL. The G=6(G=3TREN) displayed specific transfection at 1 and 5 μg/mL, non-specific at 10 μg/mL and toxicity at ≧50 μg/mL. G=6EA(G=3) showed no real transfection ability and significant toxicity starting at 50 μg/mL. G=6(G=4TREN) also showed no significant transfection capabilities but was toxic at ≧50 μg/mL. G=4EA(G=3) shows specific transfection effects at 50 μg/mL and toxicity at 100 μg/mL. G=4EA(G=2) showed some specific transfection at 1 μg/mL and 50 μg/mL and toxicity at 400 μg/mL. G=5(G=3TREN) showed very slight specific transfection at 50 μg/mL and toxicity increasing from 100 μg/mL.

In MDCK cells Lipofectamine showed some specific knockdown and no significant toxicity. The G=6(G=3TRIS) showed no specific transfection ability and no toxicity. The G=6(G=3TREN) showed no specific transfection ability, however displayed toxicity at ≧50 μg/mL. The G=6EA(G=3) also showed no specific transfection ability and toxicity at ≧50 μg/mL. The same was found for G=6(G=4TREN). G=4EA(G=3) showed specific transfection at 100 μg/mL, however toxicity began to be noticeable at 50 μg/mL and increased as concentration got higher. G=4EA(G=2) showed no specific transfection ability and toxicity at starting at 200 μg/mL and increasing at 400 mg/mL. G=5(G=3TREN) showed no specific transfection and toxicity starting at 100 μg/mL and increasing with higher concentrations.

The amine surfaces on the shell of the core-shell structures appear to be necessary for transfection (likely for the ability to bind the siRNA). However, the larger the core shell structures the more toxic to the cells. In fact there was little transfection seen with the largest structures (G=6 cores): this may be due either to the increased toxicity or possibly they need to be tested at a lower concentration since the high number of amine surface groups can more efficiently carry the short siRNAs. G=4EA(G=3) showed the best specific transfection for both cell lines in these studies. This size structure may represent a balance between ability to efficiently carry the siRNA and having lower toxicity. It is likely, however, that individual transfection agents will interact differently with different cell lines, so that it will be necessary to optimize specific conditions for each cell line even after a general carrier is found.

### EXAMPLE III

### Transfection/Western Blot Methods & Results

### Methods

### Transfection

Lyophilized core-shell tecto(dendrimers) of Formula I [G=4EA(G=2), G=5(G=3TREN), and G=4(G=3TREN)] were brought up to 250 μl in MEM (10% FBS) in concentrations ranging from 50-450 μg/mL. In a separate Eppendorf tube, Cyclophilin B siRNA [Human PPIB; siGENOME duplex (Dharmacon, Inc.)] was brought up to 250 μL in MEM (10% FBS) for a final concentration of 150 nM. Both were allowed to incubate at RT for 15 mins. before mixing together and incubating for an additional 20 mins. Another 500 μL of media was added to each tube after incubation, bringing the total volume to 1 mL. This mixture was then added to 85% confluent HEK 293 or MDCK cells whose media had been completely aspirated. The cells were incubated with the dendrimer-siRNA complexes for 6 hours before replacing with fresh media. The cells were fed 48 hours later, and then harvested after 72 hours. The tissue culture plates were rinsed with PBS, then scraped in 150 μL Western Lysis Buffer (15 mM of TRIS-HCL pH 7.4-8.0, 150 mM of NaCl, 1% of Triton X-100, and 1 mM of NaVO₄) and transferred to Eppendorf tubes. The samples were then vortexed and frozen at −20° C. until protein analysis.

Lipofectamine™ 2000 (Invitrogen) transfections were performed per the manufacturer's protocol. Basically, the same procedure as above was performed, however the media during complex formation was free from FBS and antibiotics. Complexes were formed with 2 μg/mL of Lipofectamine 2000.

### Protein Quantitation

Protein samples were thawed and vortexed, then centrifuged at 12K rpm. Samples were analyzed for protein content using the BioRad™ Protein Assay (BioRad) per manufacturer's protocol. Basically, 2 μL of protein sample were added to a 96 well microplate, followed by 200 μL of diluted BioRad™ reagent. The plate was read at 570 nm on a Multiskan MCC/340 microplate reader (ThermoLabsystems). BSA was used for the standard. Calculations were performed on the resulting data to determine protein quantitation of the samples.

### Western Blots

Twenty five micrograms of protein samples were run on 15%/5% SDS PAGE. The gels were run at 30 mA per gel. Following electrophoresis, the gels were assembled in a gel transfer apparatus and transferred to nitrocellulose membrane in 2.2 g/L of sodium bicarbonate at 200 mA for 2 hours. The membranes were then removed, probed with Ponceau Red to monitor transfer efficacy, rinsed with TBS, and blocked in a 5% milk solution for 1 hour. After blocking, the membranes were incubated at RT with anti-Cyclophilin B antibody (1:3000 dilution) for 2 hours (Abcam, Inc.), followed by 2×5 min. rinses with TBS+0.05% Tween. Alkaline phosphatase-conjugated anti-rabbit secondary antibody (1:5000 dilution) was then incubated with the membranes for 1 hour, followed by 3×5 min. rinses with TBS+0.05% Tween. The membranes were then developed using 1-Step™ NBT/BCIP solution from Pierce. For a loading control, the membranes were incubated with anti-β-actin antibody (1:3000 dilution) for 1 hour (Abcam, Inc.). Alkaline phosphatase-conjugated anti-mouse antibody (1:5000 dilution) was used as the secondary antibody as per the anti-rabbit described above. Washes were performed as described above, as well. Images were captured digitally and analyzed for band density using ImageJ software (NIH).

### Results

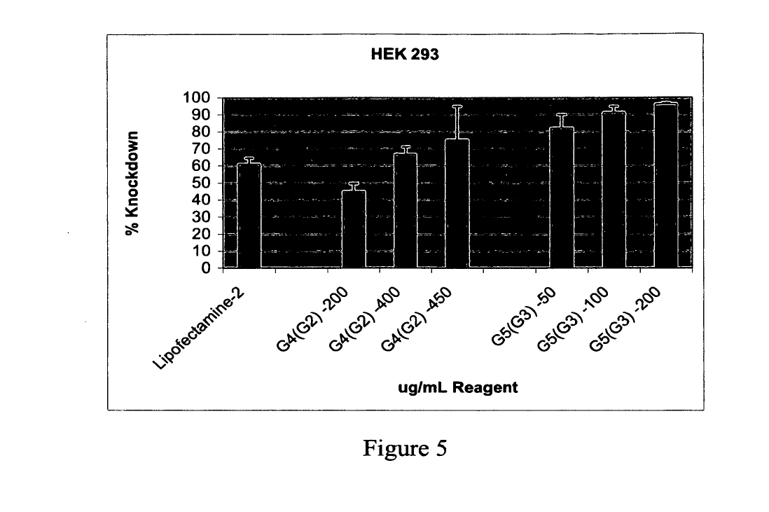

The results from transfecting siRNA into both HEK 293 and MDCK cells using G=4EA(G=2), G=5(G=3TREN), and G=4(G=3TREN) core-shell tecto(dendrimers) are shown in FIGS. 5, 6, and 7. In the HEK 293 cells, Lipofectamine™ 2000, a commercially available transfection agent, resulted in 61% knockdown of Cyclophilin B protein. The G=4EA(G=2) dendrimers had similar results, depending on concentration of dendrimer used. The G=5(G=3TREN) dendrimers significantly increased the knockdown of Cyclophilin B protein compared to Lipofectamine 2000, reaching 96% knockdown at 200 μg/mL. See FIG. 5.

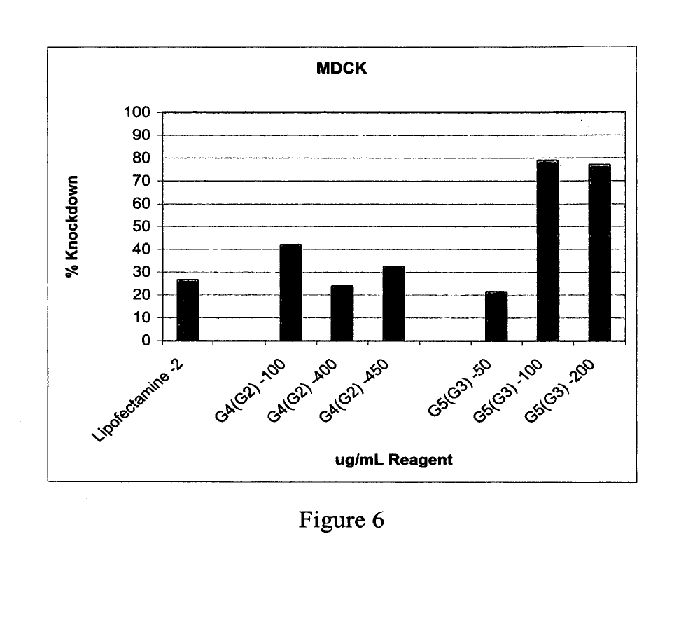

In MDCK cells, a cell line that is much more difficult to transfect, Cyclophilin B protein knockdown mediated by Lipofectamine™ 2000 delivery was 27% (see FIG. 6). The G=4EA(G=2) dendrimers showed similar results at higher concentrations used, with an increase in knockdown to 42% when the concentration was lowered to 100 μg/mL. Using the G=5(G=3TREN) dendrimers to deliver the siRNA, a substantial increase in protein knockdown was seen at both 100 and 200 μg/mL (78% knockdown), with an increase almost 3 times that of Lipofectamine 2000. See FIG. 6.

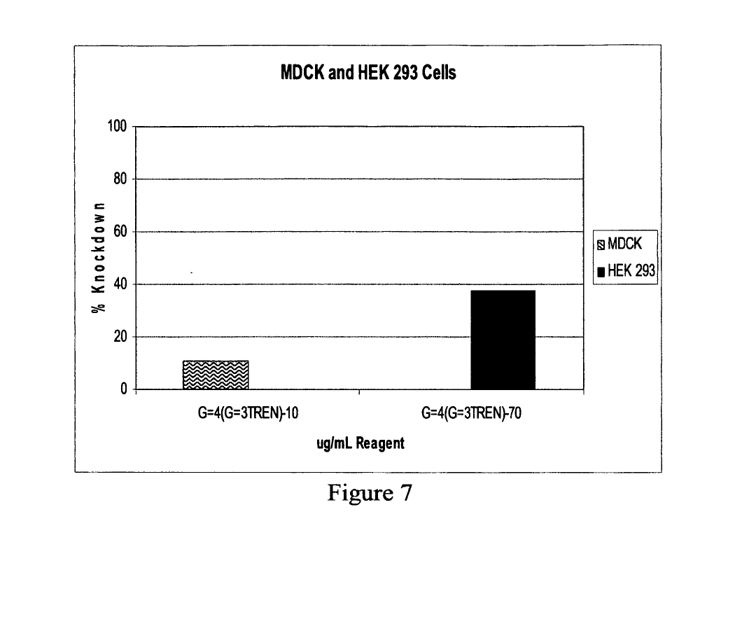

Results from transfecting the G=4(G=3TREN) PEHAM core-shell tecto(dendrimers) into MDCK and HEK 293 cells are shown in FIG. 7. In the HEK 293 cells, 37% Cyclophilin B protein knockdown was seen at 70 μg/mL. In MDCK cells, 11% protein knockdown was obtained using 10 μg/mL. No observable toxicity was noted.

Core-shell tecto(dendrimers) then may be used to transfect siRNA into both easy and hard to transfect cell lines (HEK 293 and MDCK as shown here), resulting in substantial knockdown of the targeted protein as determined by Western blot.

Although the invention has been described with reference to its preferred embodiments, those of ordinary skill in the art may, upon reading and understanding this disclosure, appreciate changes and modifications which may be made which do not depart from the scope and spirit of the invention as described above or claimed hereafter.

1. A core-shell tecto(dendritic polymer) structure of the formula:
[C-(TF)ₙ]*[S-(TF)ₘ]ₓ Formula I
wherein:
[C] is a core dendritic polymer having (TF) groups present;
(TF) means a terminal functionality, where n≧1, which, if n is greater than 1, then (TF) may be the same or a different moiety;
n means the number of surface groups from 1 to the theoretical number possible for [C];
[S] is a shell dendritic polymer having (TF) groups present;
(TF) means a terminal functionality, which, if m is greater than 1, then (TF) may be the same or a different moiety;
m means the number of surface groups from 1 to the theoretical number possible for [S];
x means the number of [S] entities that surround [C] which are from 1 to the theoretical number possible for the (TF) present on [C];
* means a covalent bond; and
provided that both [C] and [S] may not be simultaneously PAMAM; and
[C] may not be a G=4 PAMAM [(C)=EDA; (TF)=NH₂], where [S] is G=1 PEHAM [(C)=TMPTGE; (IF1)=OH; (BR1)=DEIDA; (TF)=Ethyl ester];
and [C] may not be a G=2 PEHAM [(C)=TMPTGE; (IF1)=OH; (BR1)=DEIDA; (BR2)=TREN; (TF)=NH₂], where [S] is G=1 PEHAM [(C)=TMPTGE; (IF1)=OH; (BR1)=DCEA; (TF)=Ethyl ester].

2. The core-shell tecto(dendritic polymer) structure of Formula I as defined in claim 1 wherein one or more biologically active materials are associated with the core-shell tecto(dendritic polymer).

3. The core-shell tecto(dendritic polymer) structure of Formula I as defined in claim 1 wherein one or more pseudo(dendritic polymers) are [C] or [S].

4. The core-shell tecto(dendritic polymer) structure of Formula I as defined in claim 1 wherein one or more [S] are dendrons.

5. The core-shell tecto(dendritic polymer) structure of Formula I as defined in claim 1 wherein one or more nucleic acids are associated with the core-shell tecto(dendritic polymer) that together form a construct.

6. The construct of claim 5 wherein the nucleic acid is single stranded (ss)DNA, RNA, PNA, LNA, and all double stranded (ds) combinations of these single stranded forms, including from any source (synthetic or naturally isolated) and any, where the sense and/or anti-sense strand nucleic acid are conjugated to the dendritic polymer.

7. The construct of claim 6 comprising a length from the smallest oligonucleotides (3 nucleotides) to whole chromosomes, including small hairpin RNA (shRNA), and aptamers, both unmodified and modified nucleic acids [on the backbone, bases, termini [3′ or 5′)], or combinations of these modifications.

8. The construct of claim 6 or 7 wherein the number of nucleotides are from about 18-30.

9. The construct of claim 5 wherein the sense and/or anti-sense strand nucleic acid are conjugated to the core-shell tecto(dendritic polymer).

10. The construct of claim 5 wherein the nucleic acid has modifications at the 5′ end, 3′ end, of the backbone, or bases.

11. The core-shell tecto(dendritic polymer) structure of Formula I as defined in claim 1 wherein one or more of the (TF) groups of [S] are further derivatized.

12. The core-shell tecto(dendritic polymer) structure of Formula I as defined in claim 1 or 2 wherein [C] is a PAMAM dendrimer and [S] is a PEHAM dendrimer.

13. The core-shell tecto(dendritic polymer) structure of Formula I as defined in claim 1 or 2 wherein [C] is a PEHAM dendrimer and [S] is a PEHAM dendrimer.

14. The core-shell tecto(dendritic polymer) structure of Formula I as defined in claim 1 or 2 wherein [C] is a PEHAM dendrimer and [S] is a PAMAM dendrimer.

15. The core-shell tecto(dendritic polymer) structure of Formula I as defined in claim 1 or 2 wherein [C] is a PEHAM dendrimer and [S] is a dendron.

16. The core-shell tecto(dendritic polymer) structure of Formula I as defined in claim 1 or 2 wherein [C] is a PAMAM dendrimer and [S] is a dendron.

17. The core-shell tecto(dendritic polymer) structure of Formula I as defined in claim 1 or 2 wherein the number of G of either [C] or [S] is from 1-6.

18. The core-shell tecto(dendritic polymer) structure of Formula I as defined in claim 1 or 2 wherein [C] is a dendrimer, dendrigraft, polylysine, pseudo(dendritic polymer), cleavable core, or random hyperbranched polymer and [S] is a dendrimer, dendron, dendrigraft, polylysine, or random hyperbranched polymer.

19. (canceled)

20. A formulation comprising a core-shell tecto(dendritic polymer) structure of Formula (I) as defined in claim 1 or 2, and
suitable carriers, excipients or diluents.

21. The formulation of a core-shell tecto(dendritic polymer) structure of Formula I as defined in claim 20 wherein one or more nucleic acids are associated with the core-shell tecto(dendritic polymer).

22. The formulation of claim 21 wherein the nucleic acid is single stranded (ss)DNA, RNA, PNA, LNA, and all double stranded (ds) combinations of these single stranded forms, including from any source (synthetic or naturally isolated) and any, where the sense and/or anti-sense strand nucleic acid are conjugated to the dendritic polymer.

23. The formulation of claim 22 comprising a length from the smallest oligonucleotides (3 nucleotides) to whole chromosomes, including small hairpin RNA (shRNA), and aptamers, both unmodified and modified nucleic acids, or combinations of these modifications.

24. The formulation of claim 22 or 23 wherein the number of nucleotides are from about 18-30.

25. The formulation of claim 21 wherein the sense and/or anti-sense strand nucleic acid are conjugated to the core-shell tecto(dendritic polymer).

26. The formulation of claim 21 wherein the nucleic acid has modifications at the 5′ end, 3′ end, of the backbone, or bases.

27. The formulation of claim 20 or 21 for use in diagnosis and/or therapy.

28. The formulation of claim 27 wherein the formulated construct has a pharmaceutically-acceptable carrier, excipient or diluent and increased solubility of the biologically active material, extended residence time in the body, provides higher blood concentration (AUC), an altered excretion pathway compared to biologically active material alone, and/or reduced toxicity.

29. The formulation comprising the core-shell tecto(dendritic polymer) structure of Formula I as defined in claim 20 wherein one or more nucleic acids are associated with the core-shell tecto(dendritic polymer) for use in in vitro applications for research or analysis.

30. The formulation comprising the core-shell tecto(dendritic polymer) structure of Formula I as defined in claim 20 wherein one or more nucleic acids are associated with the core-shell tecto(dendritic polymer) for use in in vivo applications for research or analysis.

31. The formulation comprising the core-shell tecto(dendritic polymer) structure of Formula I as defined in claim 20 wherein one or more nucleic acids are associated with the core-shell tecto(dendritic polymer) for use in ex vivo applications for research or analysis.

32. A method of delivering a construct of claim 5 or the formulation of claim 21 to a cell for RNAi and/or gene therapy in vivo, in vitro or ex vivo which comprises administering the construct to the cell.

33. The method of claim 32 wherein the construct is used in conjunction with other transfection agents and/or transfection enhancers.

34. The method of claim 32 wherein the core-shell tecto(dendritic polymer) structure of Formula I of claim 1 or the formulated construct of claim 21 has a positive or partially positive charge.

35. The method of delivering a construct of claim 2 or formulation of claim 20 to a cell for delivery of biologically active material to an animal or plant.

36. The method of delivering a construct of claim 2 or formulation of claim 20 to an animal which modifies the pharmacological behavior of the biologically active material.

37. The method of claim 36 wherein the construct has enhanced solubility in body fluids and pharmaceutically-acceptable solutions and suspensions.

38. The method of claim 35 wherein the core-shell tecto(dendritic polymer) structure of Formula I of claim 1 or the formulated construct of claim 20 also has a target director present.

39. The method of claim 38 or 47 wherein the target director is as an antibody, ligand, and/or receptor molecule.

40. The method of claim 32 wherein the core-shell tecto(dendritic polymer) structure of Formula I of claim 1 or the formulated construct of claim 20 also has a detection moiety present such as a dye, fluorescent moiety, radionucleotide, metal particles, chelated ions used in MRI, PET, and SPECT detection, and/or quantum dots to monitor the delivery of the construct into the cells.

41. The method of claim 32 wherein the construct is administered by standard incubation, electroporation, ballistic transfection, high pressure delivery, dermal, direct injection, or any other suitable method.

42. The method of claim 35 wherein an effective amount of the construct is administered to an animal in need of such treatment containing a formulation of any one of claims 20-23, 25, 26, or 28-31 of a core-shell tecto(dendritic polymer) structure of Formula I as defined in claim 20.

43. The method of claim 35 wherein the construct is administered by an oral route, ampoule, intravenous injection, intramuscular injection, transdermal application, intranasal application, intraperitoneal administration, subcutaneous injection, ocular application, as wipes, sprays, gauze or other means for use at a surgical incision, near scar formation sites, or site of tumor growth or removal, or near or within a tumor.

44. The method of claim 32 or 43 wherein the effective amount of the construct administered to the animal is the same as previously known or less to obtain the same effect.

45. A kit comprising a core-shell tecto(dendritic polymer) structure of Formula I as defined in any one of claims 1-31 for use in an assay as a biomarker reagent, molecular probe, transfection reagent, or environmental assay reagent together with any other components required for such assay either in separate containers or obtained separately and with instructions on use.

46. The method of claim 33 wherein the core-shell tecto(dendritic polymer) structure of Formula I of claim 1 or the formulated construct of claim 21 has a positive or partially positive charge.

47. The method of claim 32 wherein the core-shell tecto(dendritic polymer) structure of Formula I of claim 1 or the formulated construct of claim 20 also has a target director present.

48. The method of claim 35 wherein the core-shell tecto(dendritic polymer) structure of Formula I of claim 1 or the formulated construct of claim 20 also has a detection moiety present such as a dye, fluorescent moiety, radionucleotide, metal particles, chelated ions used in MRI, PET, and SPECT detection, and/or quantum dots to monitor the delivery of the construct into the cells.
